(19) United States
(12) Patent Application Publication
(10) Pub. No.: US 2024/0229098 A1
(43) Pub. Date: Jul. 11, 2024

(54) BIOLUMINOGENIC ASSAY FOR DRUG-RESISTANCE BACTERIA DETECTION

(71) Applicant: The Board of Trustees of the Leland Stanford Junior University, Stanford, CA (US)

(72) Inventors: Jianghong RAO, 936 Dennis Drive, CA (US); Tingting DAI, Menlo Park, CA (US); Jinghang XIE, Fremont, CA (US); Ran MU, Stanford, CA (US)

(21) Appl. No.: 18/560,486

(22) PCT Filed: May 18, 2022
(86) PCT No.: PCT/US2022/029875

§ 371 Date: Nov. 13, 2023

Related U.S. Application Data
(60) Provisional application No. 63/190,473, filed on May 19, 2021.

(51) Int. Cl. C12Q 1/04; C07D 501/18; C12Q 1/66

(52) U.S. Cl. CPC C12Q 1/04; C07D 501/18; C12Q 1/66

(57) ABSTRACT

Caged luciferin-based probes become a luciferase substrate emitting bioluminescence upon β-lactamase/esterase activation. The inclusion of a cephalosporin moiety renders the probe capable of being used for the detection of a wide-range of β-lactamases and β-lactamase-expressing bacteria. Embodiments of a rapid high-throughput assay for the identification of β-lactamase-expressing bacteria is made possible by the use of such probes. In some embodiments the cephalosporin is substituted by a carbapenem moiety to generate carbapenem-caged luciferin carbapenem-cleavable probes capable of being used for the detection of a wide-range of carbapenem-expressing bacteria. Accordingly embodiments of a rapid high-throughput assay for the identification of carbapenem-expressing bacteria is made possible by the use of these probes.

### CROSS-REFERENCE TO RELATED APPLICATION

This application claims the benefit of U.S. Provisional Application No. 63/190,473 filed on May 19, 2021, titled “BIOLUMINOGENIC ASSAY FOR DRUG-RESISTANCE BACTERIA DETECTION” the entire disclosure of which is incorporated herein by reference.

### STATEMENT REGARDING FEDERALLY SPONSORED RESEARCH OR DEVELOPMENT

This invention was made with Government support under contract Al125286 awarded by the National Institutes of Health. The Government has certain rights in the invention.

### TECHNICAL FIELD

The present disclosure is generally related to compositions comprising a β-lactamase- or carbapenemase-sensitive luciferin-based probes and methods of synthesis thereof. The present disclosure is also generally related to a method of detecting a β-lactamase- or carbapenemase-resistant bacterial strain.

### BACKGROUND

Since the discovery of penicillin in 1928, β-lactam antibiotics (e.g., penicillin, cephalosporins, carbapenems and monobactams) retain a central role in treating bacterial infections, constituting 60% of worldwide antibiotic usages (Fleming, A. Br. J. Exp. Pathol. 10, 226-236 (1929)). However, bacterial resistance to β-lactam antibiotics has also emerged, which has been accelerated by their abuse and misuse in veterinary and human medicine. To date, the most common resistance mechanism for this category of antibiotics, either acquired horizontally or vertically, is the production of β-lactamases such as penicillinases, extended-spectrum β-lactamases (ESBLs), AmpC-type β-lactamases (ACBLs), and carbapenemases. These enzymes could hydrolyze the amide bond of the β-lactam ring and inactivate the drugs (Tooke et al. J. Mol. Biol. 431, 3472-3500 (2019)). Over the past decades, unique β-lactamases were continuously discovered, and the number grew almost exponentially (Bush, K. Antimicrob. Agents Chemother. 62, e01076-18 (2018)). To optimize the use of β-lactam antibiotics and promote the antimicrobial stewardship, the implementation of new diagnostics for more rapid and sensitive detection of β-lactamase activities would be advantageous.

There are several clinically adopted diagnostics of β-lactam resistance. Culture-based methods such as double-disk synergy and combination disk tests, and automated liquid culture have been widely used as clinical standard. In spite of good sensitivity and specificity (80-95%), these methods normally require 1-2 days to generate results (Aruhomukama, D. Afr. Health Sci. 20, 1090-1108 (2020); Gazin et al., J. Clin. Microbiol. 50, 1140-1146 (2012)). Molecular diagnostics such as fluorescence in situ hybridization (FISH) and PCR have been developed for the detection of β-lactamase gene signature with high sensitivity and specificity (Jamal et al., J. Clin. Microbiol. 52, 2487-2492 (2014)). However, the assays often require pre-enrichment and isolation of the pathogenic bacteria to generate a reliable readout. Resistance predicted by genotypic analysis does not always correlate with phenotypic results, and emerging new mutations may evade their detection, giving false-negative results (Bush, K. Antimicrob. Agents Chemother. 62, e01076-18 (2018); Paterson & Bonomo Clin. Microbiol. Rev. 18, 657-686 (2005)).

Substrate-based enzyme function assays can directly reveal whether the bacteria possess the capability to destroy β-lactam antimicrobial activity in a viable status. Over the past few years, fluorescent probes have been developed to detect ESBLs/AmpC activity (Zhang et al., Angew. Chem. Int. Ed. 51, 1865-1868 (2012); Thai et al. Biosens. Bioelectron. 77, 1026-1031 (2016); Aw et al. Chem. Commun. 53, 3330-3333 (2017); Khan et al., BMC Microbiol. 14, 84 (2014)). In addition, a pH-based colorimetric assay, Carba NP, has been approved by Clinical & Laboratory Standards Institute (CLSI) for carbapenemase activities detection. However, their sensitivity is moderate and they are subject to the interference of nonspecific or autofluorescence signals from patient specimens (Tamma et al., J. Clin. Microbiol. 56, (2018)). Compared with methods using these fluorescent probes, bioluminescence detection does not use excitation light but enzyme-produced photon emission, has extremely low background and is not subject to interference of nonspecific or autofluorescence signals in patient specimens. Bioluminescence-based detection therefore promises high sensitivity and specificity.

### SUMMARY

Provide are caged luciferin-based probes that become a luciferase substrate emitting bioluminescence upon β-lactamase/esterase activation. The inclusion of a cephalosporin moiety renders the probe capable of being used for the detection of a wide-range of β-lactamases and β-lactamase-expressing bacteria. Embodiments of a rapid high-throughput assay for the identification of β-lactamase-expressing bacteria is made possible by the use of such probes. In some embodiments the cephalosporin is substituted by a carbapenem moiety to generate carbapenem-caged luciferin carbapenem-cleavable probes capable of being used for the detection of a wide-range of carbapenem-expressing bacteria. Accordingly embodiments of a rapid high-throughput assay for the identification of carbapenem-expressing bacteria is made possible by the use of these probes. The caged luciferin probes generate low levels of bioluminescence in the absence of β-lactamase or carbapenem, which increases the sensitivity of the assay method and reduces the time that may be necessary to culture the suspected antibiotic-resistant bacterial strains for a detectable result. The cascade activation of the caged luciferins of the disclosure facilitate rapid diagnosis of lactam-resistant bacterial pathogens and timely selection of appropriate treatment and prevent further spread of antibiotic resistance.

One aspect of the present disclosure encompasses embodiments of a caged bioluminescent probe comprising a luciferin moiety, an enzyme-cleavable moiety, and a caging moiety, wherein the moieties are conjugated to form the structure having the formula:

(luciferin moiety)-(enzyme-cleavable moiety)-(caging moiety),

and wherein the enzyme-cleavable moiety can be cleaved by a bacterial enzyme.

In some embodiments of this aspect of the disclosure, the caged bioluminescent probe can further comprise a linker moiety between the luciferin moiety and the enzyme-cleavable moiety.

In some embodiments of this aspect of the disclosure, the enzyme-cleavable moiety is cleavable by a β-lactamase.

In some embodiments of this aspect of the disclosure, the caging moiety can be a quencher.

In some embodiments of this aspect of the disclosure, the quencher can be a diabcyl quencher.

In some embodiments of this aspect of the disclosure, the caged bioluminescent probe can have the formula:

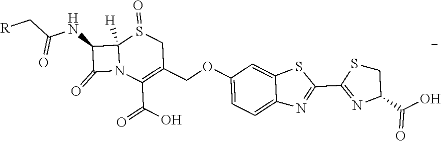

wherein R is

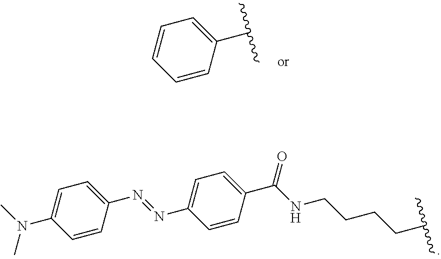 [SMILES: *c1ccccc1 ; *CCCCNC(=O)c1ccc(/N=N/c2ccc(N(C)C)cc2)cc1]

In some embodiments of this aspect of the disclosure, the enzyme-cleavable moiety can be cleavable by a carbapenemase.

In some embodiments of this aspect of the disclosure, the caged bioluminescent probe can have the formula:

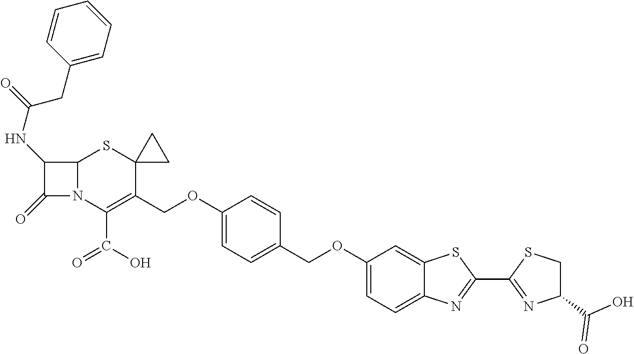 [SMILES: O=C(Cc1ccccc1)NC1C(=O)N2C(C(=O)O)=C(COc3ccc(COc4ccc5nc(C6=N[C@@H](C(=O)O)CS6)sc5c4)cc3)C3(CC3)SC12]

wherein the linker has the structure

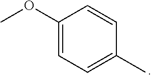

In some embodiments of this aspect of the disclosure, the caged bioluminescent probe can have the formula:

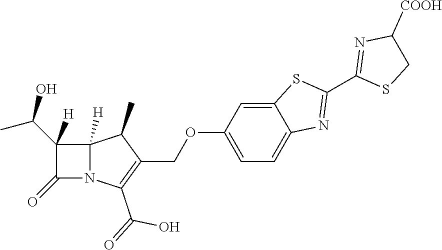 [SMILES: C[C@@H](O)[C@H]1C(=O)N2C(C(=O)O)=C(COc3ccc4nc(C5=NC(C(=O)O)CS5)sc4c3)[C@H](C)[C@H]12]

In some embodiments of this aspect of the disclosure, the caged bioluminescent probe can further comprise a linker moiety between the luciferin moiety and the enzyme-cleavable moiety

In some embodiments of this aspect of the disclosure, the caged bioluminescent probe can have the formula:

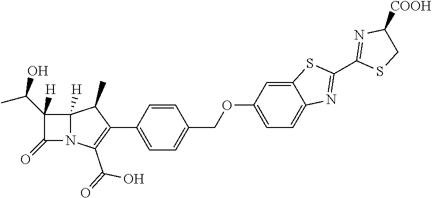 [SMILES: C[C@@H](O)[C@H]1C(=O)N2C(C(=O)O)=C(c3ccc(COc4ccc5nc(C6=N[C@@H](C(=O)O)CS6)sc5c4)cc3)[C@H](C)[C@H]12]

wherein the linker has the structure

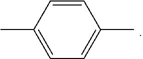

Another aspect of the present disclosure encompasses embodiments of a method of identifying a bacterial strain resistant to a β-lactam antibiotic or a carbapenem antibiotic, wherein said method can comprise contacting a population of bacteria with a caged bioluminescent probe cleavable by either a β-lactamase or a carbapenemase, adding luciferinase; and measuring an emitted bioluminescent signal, wherein a detected emitted bioluminescent signal indicates that the bacterial strain has a β-lactamase or a carbapenemase activity.

In some embodiments of this aspect of the disclosure, the caged bioluminescent probe can comprise a luciferin moiety, an enzyme-cleavable moiety, and a caging moiety, wherein the moieties are conjugated to form the structure having the formula:

(luciferin moiety)-(enzyme-cleavable moiety)-(caging moiety),

and wherein the enzyme-cleavable moiety can be cleaved by a bacterial enzyme.

In some embodiments of this aspect of the disclosure, the caged bioluminescent probe can comprise a linker moiety between the luciferin moiety and the enzyme-cleavable moiety.

In some embodiments of this aspect of the disclosure, the enzyme-cleavable moiety is cleavable by a β-lactamase or by a carbapenemase.

In some embodiments of this aspect of the disclosure, the caging moiety can be a quencher.

In some embodiments of this aspect of the disclosure, the caged bioluminescent probe can have the formula:

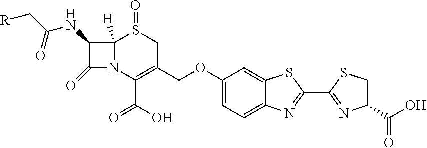

wherein R is

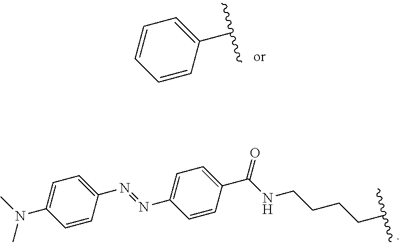 [SMILES: *c1ccccc1 ; *CCCCNC(=O)c1ccc(/N=N/c2ccc(N(C)C)cc2)cc1]

In some embodiments of this aspect of the disclosure, the caged bioluminescent probe can have the formula:

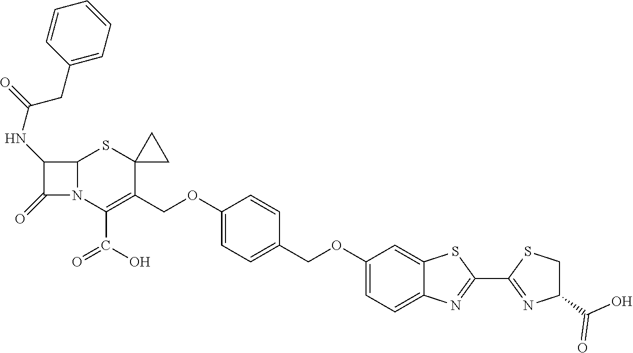 [SMILES: O=C(Cc1ccccc1)NC1C(=O)N2C(C(=O)O)=C(COc3ccc(COc4ccc5nc(C6=N[C@@H](C(=O)O)CS6)sc5c4)cc3)C3(CC3)SC12]

wherein the linker has the structure

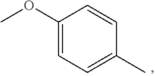

and wherein the enzyme-cleavable moiety can be cleaved by a carbapenemase.

In some embodiments of this aspect of the disclosure, the caged bioluminescent probe can have the formula:

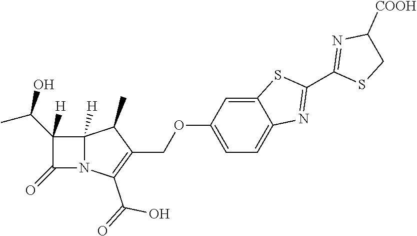 [SMILES: C[C@@H](O)[C@H]1C(=O)N2C(C(=O)O)=C(COc3ccc4nc(C5=NC(C(=O)O)CS5)sc4c3)[C@H](C)[C@H]12]

In some embodiments of this aspect of the disclosure, the caged bioluminescent probe can further comprise a linker moiety between the luciferin moiety and the enzyme-cleavable moiety

In some embodiments of this aspect of the disclosure, the caged bioluminescent probe can have the formula:

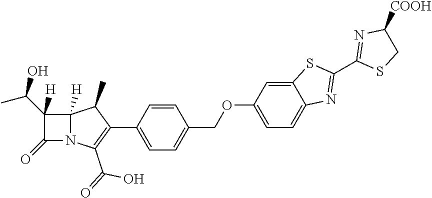 [SMILES: C[C@@H](O)[C@H]1C(=O)N2C(C(=O)O)=C(c3ccc(COc4ccc5nc(C6=N[C@@H](C(=O)O)CS6)sc5c4)cc3)[C@H](C)[C@H]12]

wherein the linker can have the structure

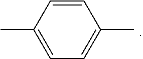

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1. Bioluminescent substrates for the β-lactamase detection.

FIG. 2 illustrates the synthesis of D-Bluco.

FIG. 3 illustrates the synthesis of Am-Bluco.

FIG. 4 illustrates the synthesis of CP-luc.

FIG. 5 illustrates the synthesis of CPL-luc.

FIG. 6 illustrates the synthesis of O-Bluco.

FIGS. 7A-7C illustrate the characterization of D-Bluco with recombinant β-lactamase.

FIG. 7A shows longitudinal monitoring of fluorescence enhancement of D-Bluco (1 μM) with or without TEM-1 (100 nM) in PBS (pH=7.4) at r.t. (Ex-330/Em-530).

FIG. 7B shows a fluorescence emission spectrum of D-Bluco (1 μM) incubated with TEM-1 (100 nM) for 40 min at r.t.; a.u. indicates arbitrary unit.

FIG. 7C shows the bioluminescent signal of Bluco or D-Bluco (10 μM) incubated with or without TEM-1 (20 nM) for 15 min in PBS (pH=7.4) at r.t. RLU indicates relative light units.

FIG. 8 illustrates enzymatic studies of D-Bluco and O-Bluco treating with or without TEM-1.

FIGS. 9A and 9B illustrates bioluminescent studies of D-Bluco and O-Bluco treating with various β-lactamases. 10 μM of Bluco, D-Bluco and O-Bluco incubated with 20 nM TEM-1 for 15 min in PBS at r.t. The bioluminescent signals for each compound was evaluated with or without the addition of TEM-1 by a luminator. FIG. 9A shows the relative bioluminescent signal of O-Bluco and FIG. 9B shows D-Bluco incubated with different concentrations of enzymes for 2 h in r.t. The relative bioluminescent was calculated by removing background signal in PBS.

FIGS. 10A and 10B illustrate bioluminescent detection of D-Bluco and O-Bluco with E. coli expressing TEM-1 bla: FIG. 10A, 10 μM of D-Bluco or FIG. 10B, O-Bluco were incubated with different concentrations of E. coli expressing TEM-1 bla for 60 min in PBS (pH=7.4). Then bioluminescent reagent was added and BLI signal was recorded. Relative bioluminescent signal represents the difference in the bioluminescent intensity with and without bacteria incubation. Data represents two replicates and 2 repeats. N=4. The correlation (r) between relative bioluminescent signal and number of cells were calculated. E. coli labeled with the corresponding probe was included as the negative control.

FIGS. 11A-11C illustrate detection of clinical isolates spiked in urine with D-Bluco a) Workflow for clinical sample detection b) Bioluminescent response for different concentrations of S. marcescens (SME) or c) E. coli/OXA-48 spiked into synthetic urine samples following the incubation with D-Bluco (10 μM) at 37° C. for an hour. Bioluminescent signal was measured and compared to that of E. coli (10⁶ c.f.u/mL). Statistical significance was calculated using the unpaired two-tailed Student's t test (** p<0.01, **** p<0.0001).

FIG. 12A and 12B illustrate HPLC analysis of O-Bluco and D-Bluco with or without TEM-1 bla. 1 μM O-Bluco or D-Bluco incubated with PBS or with TEM-1 (100 nM) for 60 min, then analyzed by HPLC at 320 nm.

FIG. 13A and 13B illustrate fluorescent emission spectra of different concentrations of D-Bluco (FIG. 13A), or O-Bluco (FIG. 13B) incubated with or without TEM-1 Bla (100 nM) in PBS (pH=7.4) at r.t. λ_{excitation}=330 nm λₑₘᵢₛₛᵢₒₙ=530 nm.

FIG. 14 illustrates D-Bluco (0.1, 1, 5, 10 μM) was incubated with TEM-1 (20 nM) at r.t. for 15 min before the addition of bioluminescent assay reagents for BLI detection. Statistical significance was calculated using the unpaired two-tailed Student's t test (** p<0.0021, *** p<0.0002, ns: not significant). The study was duplicated at room temperature with PBS (pH=7.4) as the buffer. Data were collected on a SpectraMax iD3 multimode microplate reader. RLU: relative light units.

FIG. 15 illustrates 10 μM D-Bluco was incubated with 20 nM TEM-1 bla for 5, 15, and 45 min before adding bioluminescent assay reagents for BLI detection. Statistical significance was calculated using the unpaired two-tailed Student's t test (** p<0.01. **** p<0.0001).

FIG. 16A and 16B illustrate the optimization for bioluminescent study. Different concentration of Enzyme CoA (1, 10, 100 μM) was incubated with 10 μM D-Bluco for 15 min before adding bioluminescent assay reagents for BLI detection. FIG. 16A: Bioluminescent intensity was monitored over 10 min. FIG. 16B: The bioluminescent intensity at 0 min (right after the addition of bioluminescent reagent).

FIG. 17 illustrates D-Luciferin incubated with TEM-1 bla and IMP-1. 10 μM D-Luciferin incubated with or without TEM-1bla and Imp-1 carbapenemase (10, 20, 50, 100 nM) in PBS (pH=7.4) for 15 min. Bioluminescent signal was measured after the addition of bioluminescent reagent. Statistical significance was calculated using the unpaired two-tailed Student's t test (** p<0.01, **** p<0.0001).

FIG. 18 illustrates the bioluminescence intensity of D-luciferin at different concentrations.

FIG. 19A and 19B illustrate 10 μM of D-Bluco (FIG. 19A) or O-Bluco (FIG. 19B) were incubated with different concentrations of E. coli expressing IMP-1 for 60 min in PBS (pH=7.4). Then bioluminescent reagent was added and BLI signal was recorded. Relative bioluminescent signal represents the difference in the bioluminescent intensity with and without bacteria incubation. Data represents 2 replicates and 2 repeats. N=4. The correlation (r) between relative bioluminescent signal and number of cells were calculated. E. coli labeled with the corresponding probe was included as the negative control.

FIGS. 20A-20E illustrates validation of bacteria concentration in the plate assay.

FIG. 20A shows the workflow for the validation process (generated via Biorender).

FIG. 20B shows the relative bioluminescence intensity of D-Bluco

FIG. 20C shows O-Bluco incubated with E. coli expressing TEM-1 bla at different concentrations. The signal generated with D-Bluco and O-Bluco incubated with E. coli only was served as the background and was removed to generate the relative bioluminescent signal.

FIG. 20D and 20E show representative images of the plate to validate the c.f.u count in the assay.

FIG. 21 illustrates intermolecular quenching of D-Bluco. D-Bluco was mixed with D-luciferin (concentration ranges from 1 nM to 10000 nM). The concentration of D-Bluco was adjusted so that the final concentration for the D-Bluco and D-luciferin equals 10 μM. The bioluminescent signal was then recorded. Statistical significance was calculated using the unpaired two-tailed Student's t test (** p<0.01, **** p<0.0001).

FIG. 22 illustrates fluorescence emission spectra of CP-Luc (1 mM) in PBS before (red line) and after (black line) IMP-1 and TEM-1 treatment.

FIGS. 23A and 23B illustrate bioluminescence detection of β-lactamase with clinic isolates by D-Bluco.

FIG. 23A shows an inhibitory study with clavulanate (2 mg/mL) and avibactam (2 mg/mL) in the presence of D-Bluco (10 μM) with E. coli/TEM. E. coli/TEM-1 treated with D-Bluco exhibited a 260-fold increase of bioluminescent intensity over PBS, which was arbitrarily set as 1 to normalize the test samples and show percentage inhibition.

FIG. 23B shows D-Bluco (10 μM) was incubated with different concentrations of E. coli or E. coli expressing TEM (E. coli/TEM). The BLI signal was monitored over 4 hrs at r.t in PBS (pH=7.4). Statistical significance was calculated using the unpaired two-tailed Student's t test (* p<0.0332, ** p<0.0021, *** p<0.0002, ns: not significant). RLU indicates relative light units.

FIGS. 24A and 24C illustrates a RAPID BLI test for clinical isolates of UTI.

FIG. 24A shows RAPID BLI assay workflow for clinical sample detection.

FIG. 24B shows bioluminescent intensity of 102, 103, 104 cfu/mL of β-lactamases-expressing clinic isolates after incubation with D-Bluco (10 μM) in diluted urine. From left to right: 1) Enterobacter cloacae/IMI, 2) E. coli/TEM, 3) K. pneumoniae/KPC, 4) E. coli/NDM, 5) E. cloacae/AmpC. c) Bioluminescent intensity of 105 cfu/mL E. coli and 103 cfu/mL K. pneumoniae/KPC. The modified rapid BLI protocol is applied. The signal of D-Bluco in PBS was subtracted before plotting. The working concentration of D-Bluco was 10 μM. All the studies were duplicated at room temperature in PBS (pH=7.4). Statistical significance was calculated using the unpaired two-tailed Student's t test (** p<0.0021, *** p<0.0002, **** p<0.0001, for comparison with 105 cfu/mL E. coli) (** p<0.0021, **** p<0.0001, ns: not significant). RLU indicates relative light units. Dotted line represents 3SD of the negative control. Part of the image is adapted from Server Medical Art.

FIG. 25 illustrates the effect of TEM-1 bla (TEM-1) or IMP-1 carbapenemase on the bioluminescent signal of D-luciferin. D-Luciferin (10 μM) was incubated without or with TEM-1 bla or IMP-1 carbapenemase (10, 20, 50, 100 nM) in PBS (pH=7.4) for 15 min. The study was duplicated at room temperature with PBS (pH=7.4). Statistical significance was calculated using the unpaired two-tailed Student's t test (ns: not significant). The study was duplicated at room temperature with PBS (pH=7.4) as the buffer. Data were collected on a SpectraMax iD3 multimode microplate reader.

FIG. 26 illustrates bioluminescent enhancement of 10⁶ cfu/mL of clinic isolates E. coli/TEM by incubation with different concentrations of CHAPS for 15 min. D-Bluco was subsequently added for another 15 min incubation before bioluminescent measurement. The study was duplicated at room temperature with PBS (pH=7.4) as the buffer. The working concentration of D-Bluco was 10 μM. The Data was collected on a TurnerBiosystem luminometer.

### DETAILED DESCRIPTION

This disclosure is not limited to particular embodiments described, and as such may. of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, microbiology, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C., and pressure is at or near atmospheric. Room temperature is defined as 20-23° C. Standard pressure is defined as 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a.” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent. Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

### Abbreviations

ESBL, extended broad-spectrum β-lactamases; TFAA, Trifluoroacetic anhydride; MLB, metallo-β-lactamase; CRE, carbapenem-resistant enterobacteriaceae; PBS, Phosphate-buffered saline; CPBA, chloroperoxybenzoic acid; TFAA, trifluoroacetic anhydride; TFA, trifluoroacetic acid; TIPS, SDS, sodium dodecyl sulfate

### Definitions

In describing and claiming the disclosed subject matter, the following terminology will be used in accordance with the definitions set forth below.

The term “caging group” as used herein refers to a moiety that can be employed to reversibly block, inhibit, or interfere with the activity (e.g., the biological activity) of a molecule (e.g., a polypeptide, a nucleic acid, a small molecule, a drug, and the like). Typically, one or more caging groups are associated (covalently or noncovalently) with the molecule but do not necessarily surround the molecule in a physical cage. Caging groups can be, for example, small moieties such as carboxyl nitrobenzyl, 2-nitrobenzyl, nitroindoline, hydroxyphenacyl, DMNPE, quinilones including bromoquinilones and derivatives thereof, or the like, or they can be, e.g., large bulky moieties such as a protein or a bead. Caging groups can be removed from a molecule, or their interference with the molecule's activity can be otherwise reversed or reduced, by exposure to an appropriate type of uncaging energy and/or exposure to an uncaging chemical, enzyme, or the like. The caging groups of the present disclosure may be released from the blocked or “caged” nucleoside triphophoester (uncoupled) by photolysis following two-photon excitation. The term “detectable moiety” as used herein refers to a label molecule (isotopic or non-isotopic) which is incorporated indirectly or directly into a liposomal nanoparticle according to the disclosure, wherein the label molecule facilitates the detection of the nanoparticle in which it is incorporated. Thus, “detectable moiety” is used synonymously with “label molecule”. Label molecules, known to those skilled in the art as being useful for detection, include chemiluminescent or fluorescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

The term “luciferin” as used herein refers to a light-emitting compound found in organisms that generate bioluminescence. Luciferins undergoes an enzyme-catalyzed reaction with molecular oxygen. The resulting transformation produces an excited state intermediate that emits light upon decaying to its ground state. The term may refer to molecules that are substrates for both luciferases and photoproteins.

Luciferins are a class of small-molecule substrates that react with oxygen in the presence of a luciferase (an enzyme) to release energy in the form of light. Because of the chemical diversity of luciferins, there is no clear unifying mechanism of action, except that all require molecular oxygen, which provides the needed energy. For example, but not intended to be limiting, luciferins include: firefly luciferin (fluc), is the substrate of beetle luciferases responsible for the characteristic yellow light emission from fireflies. Adenosine triphosphate (ATP) is required for light emission, in addition to molecular oxygen; Latia luciferin ((E)-2-methyl-4-(2,6,6-trimethyl-1-cyclohex-1-yl)-1-buten-1-ol formate) from the snail Latia neritoides; bacterial luciferin (FMN), a two-component system of a flavin mononucleotide and a fatty aldehyde; coelenterazine, found in a wide variety of invertebrates and fish and is the prosthetic group in the protein aequorin; dinoflagellate luciferin is a chlorophyll tetrapyrrole) and is found in some dinoflagellates; Vargulin (cypridinluciferin) is found in certain ostracods and deep-sea fish and is an imidazopyrazinone.

The term “luciferase” as used herein refers to the class of oxidative enzymes that produce bioluminescence, and is usually distinguished from a photoprotein. Luciferases are widely used in biotechnology, for bioluminescence imaging microscopy and as reporter genes, for many of the same applications as fluorescent proteins. Unlike fluorescent proteins, luciferases do not require an external light source, but do require addition of luciferin, the consumable substrate.

Luciferases have usually been found in animals, including fireflies, and many marine animals such as copepods, jellyfish, and the sea pansy. However, luciferases have been studied in luminous fungi, luminous bacteria, and dinoflagellates.

All luciferases are classified as oxidoreductases (EC 1.13.12 .-), meaning they act on single donors with incorporation of molecular oxygen. Because luciferases are from many diverse protein families that are unrelated, there is no unifying mechanism, as any mechanism depends on the luciferase and luciferin combination. However, all characterized luciferase-luciferin reactions to date have been shown to require molecular oxygen at some stage.

Luciferases can be produced in the lab through genetic engineering for a number of purposes. Luciferase genes can be synthesized and inserted into organisms or transfected into cells.

In the luciferase reaction, light is emitted when luciferase acts on the appropriate luciferin substrate. Photon emission can be detected by light sensitive apparatus such as a luminometer or modified optical microscopes. This allows observation of biological processes. Since light excitation is not needed for luciferase bioluminescence, there is minimal autofluorescence and therefore almost background-free fluorescence. Therefore, as little as 0.02 pg can still be accurately measured using a standard scintillation counter.

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations, or heat. Sometimes the absorbed photon is in the ultraviolet range, and the emitted light is in the visible range, but this depends on the absorbance curve and Stokes shift of the particular fluorophore.

The term “fluorophore” as used herein refers to any reporter group whose presence can be detected by its light emitting properties.

The term “operably linked” as used herein refers to an arrangement of elements wherein the components so described are configured so as to perform their usual function.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally-recognized pharmacopeia for use in animals, and more particularly in humans. Such pharmaceutical carriers can be liquids, such as water and oils, including those of petroleum, animal, vegetable, or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol, and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “self-immolative probe” as used herein refers to a signaling molecule covalently bound to a moiety (a “self-immolative arm” or “self-immolative linker”) such that the self-immolative arm inhibits the signaling molecule from signaling. The self-immolative arm is covalently bound to an reporter moiety such as a fluorophore of the disclosure such that the removal of a moiety by the action of an enzyme, for example, causes a destabilization of the self-immolative arm such that the self immolative arm becomes removed from the signaling molecule, allowing the signaling molecule to signal. The “self-immolative” moieties of the disclosure include such as a substrate group of an enzyme. For example, but not intended to be limiting a self-immolative linker may have attached thereon a phosphate group. A cellular phosphatase may, on contact with a probe of the disclosure, cleave the phosphate group from the linker, thereby allowing the liner to reconfigure to allow electron transfer to the fluorophore to emit a detectable signal.

The terms “quench” or “quenches” or “quenching” or “quenched” as used herein refer to reducing the signal produced by a molecule. It includes, but is not limited to, reducing the signal produced to zero or to below a detectable limit. Hence, a given molecule can be “quenched” by another molecule and still produce a detectable signal albeit the signal is greatly reduced.

The term “β-lactamase” as used herein refers to enzymes that hydrolyze the β-lactam ring of the β-lactam antibiotics. According to Ambler (Philos. Trans. R. Soc. London Ser B, (1980) 289: 321-331), β-lactamases are classified in 4 groups: A: penicillinases, including extended broad-spectrum β-lactamases (ESBLs); B: metallo-enzymes; cephalosporinases; D: oxacillinases.

Hydrolysis of the amide bond of the β-lactam ring makes the antimicrobial agents biologically inactive. Class A β-lactamases (Ambler classification) refer to serine β-lactamases, in which hydrolysis of β-lactam is mediated by serine in the active site, usually at amino acid position 70 in the alpha helix₂.

The term “carbapenemases” as used herein are a diverse group of β-lactamases that are active not only against the oxyimino-cephalosporins and cephamycins but also against the carbapenems. Carbapenemase may be a metallo-β-lactamase or a serine-β-lactamase. Broad spectrum carbapenemases can be selected from, for example, an IMP-type carbapenemases (metallo-β-lactamases), VIMs (Verona integron-encoded metallo-β-lactamases), OM (oxacillinase) group of β-lactamases, KPCs (Klebsiella pneumonia carbapenemases), CMY (Class C), SME, IMI, NMC, GES (Guiana extended spectrum), CcrA, SFC-1, SHV-38, and NDM (New Delhi metallo-β-lactamases, e.g. NDM-1) β-lactamases.

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorophore.

Spectroscopic, photochemical, biochemical, immunochemical, electrical, optical, or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

### Discussion

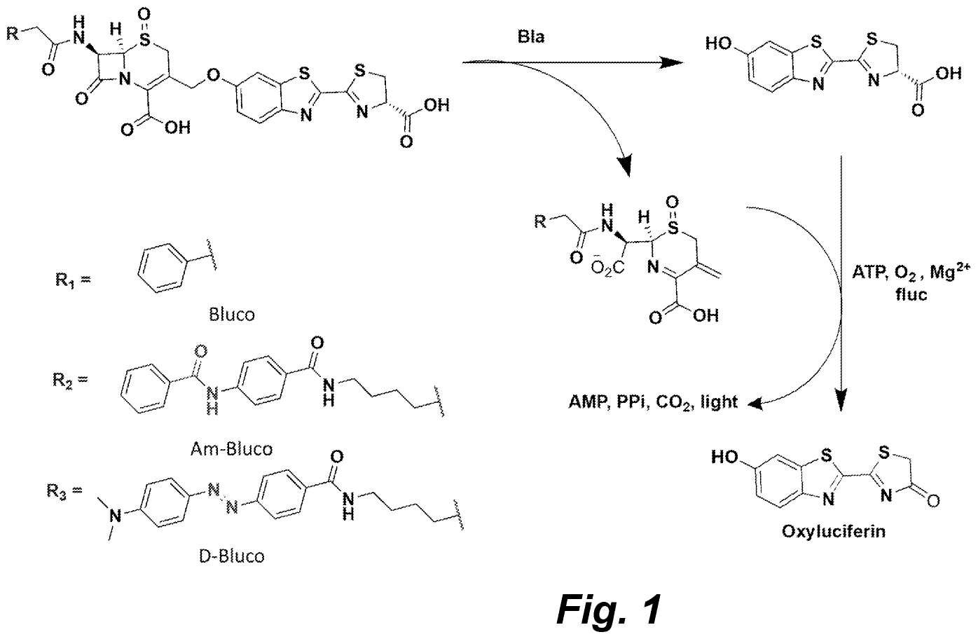

Embodiments of the present disclosure provide for caged luciferin-based probes that become a luciferase substrate emitting bioluminescence upon β-lactamase/esterase activation. The inclusion of a cephalosporin moiety renders the probe capable of being used for the detection of a wide-range of β-lactamases and β-lactamase-expressing bacteria. Embodiments of a rapid high-throughput assay for the identification of β-lactamase-expressing bacteria is made possible by the use of such probes. In some embodiments the cephalosporin is substituted by a carbapenem moiety to generate carbapenem-caged luciferin carbapenem-cleavable probes capable of being used for the detection of a wide-range of carbapenem-expressing bacteria. Accordingly embodiments of a rapid high-throughput assay for the identification of carbapenem-expressing bacteria is made possible by the use of these probes. The use of the caged luciferin probes of the disclosure have been found to generate extremely low levels of bioluminescence in the absence of a β-lactamase or carbapenem, which increases the sensitivity of the assay method and reduces the time that may be necessary to culture the suspected antibiotic-resistant bacterial strains for a detectable result. The cascade activation of the caged luciferins of the disclosure facilitate rapid diagnosis of lactam-resistant bacterial pathogens and timely selection of appropriate treatment and prevent further spread of antibiotic resistance. Probe design and synthesis. A first generation of a bioluminescent probe (BLUCO in FIG. 1) (Yao et al., Angew. Chem. Int. Ed. 46, 7031-7034 (2007)) was developed for detecting the activity of β-lactamases in Gram-negative bacteria. The 6′-hydroxy group of D-luciferin (the substrate of firefly luciferase) which is crucial for luciferase recognition was caged by an analogue of cefazolin, the first generation of cephalosporin antibiotics, which can be recognized by a broad spectrum of β-lactamases from cephalosporinases to carbapenemases. Upon β-lactamase activation, the active D-luciferin substrate is released, and bioluminescence is produced with firefly luciferase.

Bioluminescence-based probes sensitive to β-lactamase activity coupled with a simple plate-reader assay allows rapid and efficient real time monitoring of pathogenic bacteria in various specimens. In addition, the small and diffusible luciferin probes enable good bacterial penetration and mobilization throughout the organism, rendering a desirable strategy for whole organism detection without the need for lysis procedures. However, its high background signal in the absence of TEM-1 bla enzyme compromises its specificity for bacteria detection. To reduce the background signal, the compositions of the present disclosure encompass embodiments of bioluminescent probes whereby DABCYL, a broad spectrum quencher, was attached to Bluco to suppress background emission via the bioluminescence resonance energy transfer (BRET) mechanism (Xu et al., Proc. Natl. Acad. Sci. 96, 151-156 (1999); Bacart et al., Biotechnol. J. 3, 311-324 (2008); Adamczyk et al., Org. Lett. 3, 1797-1800 (2001)).

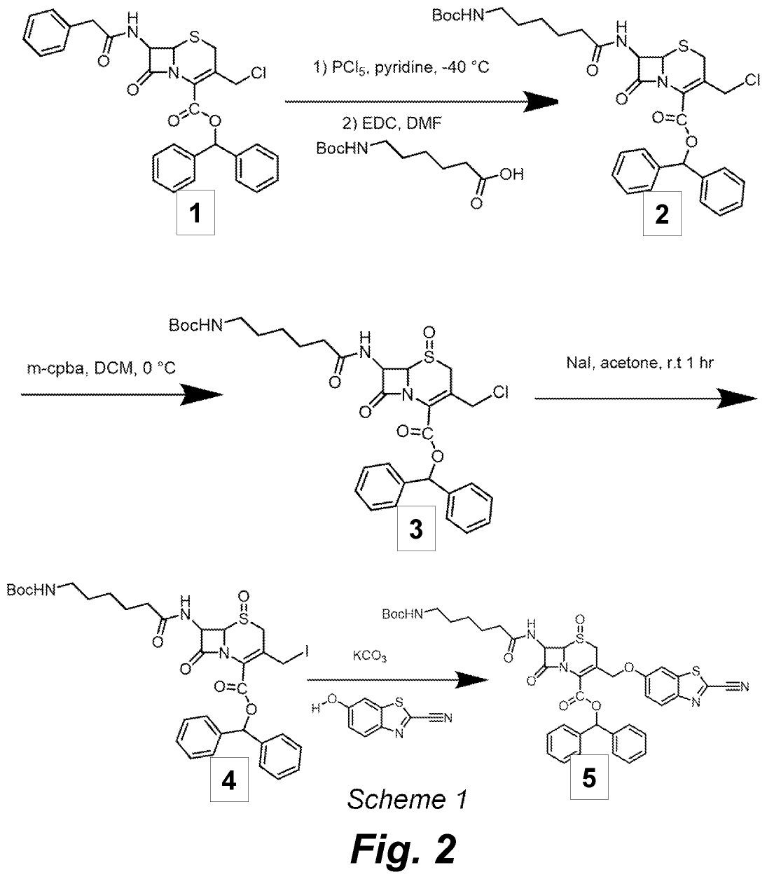

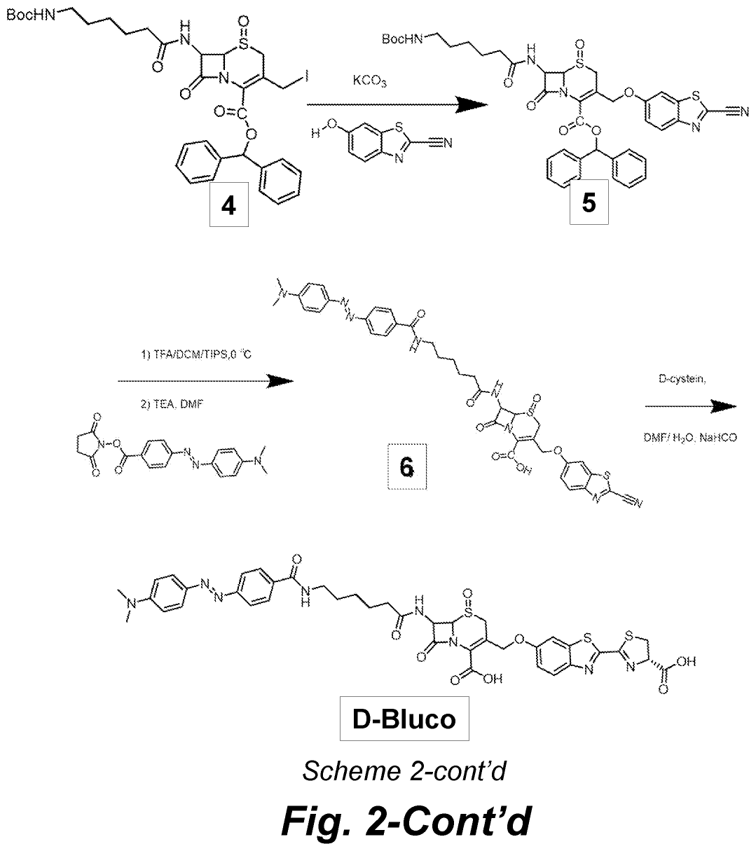

The second approach to suppressing the background was to introduce to Bluco a self-immolative linker between the cephalosporin core and the 6′ hydroxy group of D-luciferin (Miska & Geiger Biol. Chem. 369, 407-412 (1988); Li et al., Chem. Soc. Rev. 42, 662-676 (2012); Sharma et al., Org. Lett. 19, 5836-5839 (2017); Takakura et al., J. Am. Chem. Soc. 137, 4010-4013 (2015); Takakura et al., Chem.—Asian J. 6, 1800-1810 (2011)). D-Bluco is composed of a dabcyl quencher, a β-lactamase responsive cephalosporin structure and a luciferin moiety. Synthesis and purification are shown in Scheme 1, FIG. 2). and chemical characterization results are described in Example 10).

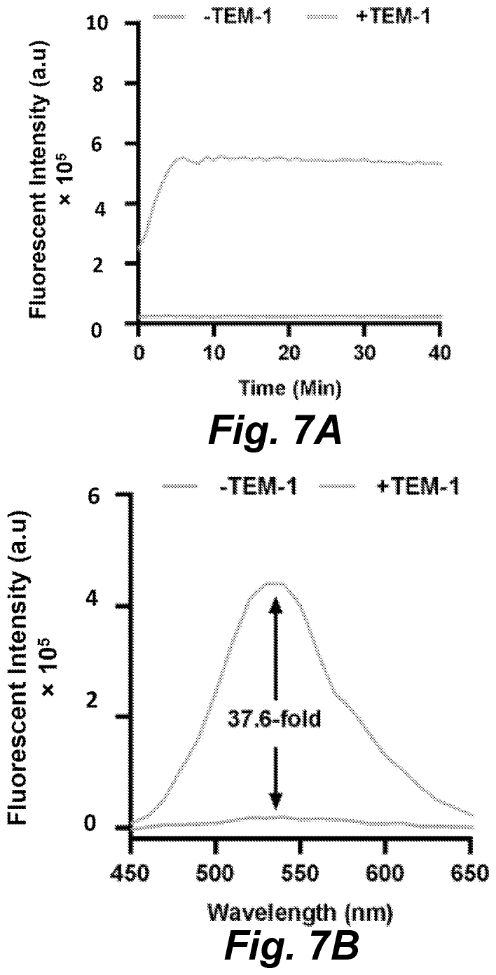

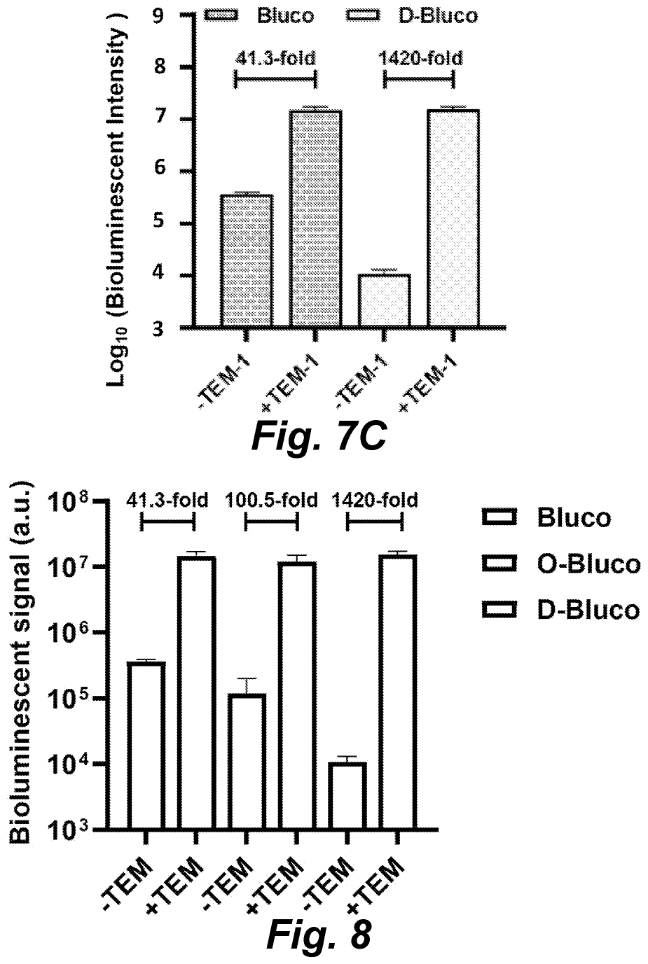

D-Bluco was evaluated as to whether it could be processed by β-lactamase to release luciferin with one of the most prevalent Ambler class A β-lactamases, TEM-1. A rapid concentration-dependent fluorescence turn on from released D-luciferin over a period of 40 minutes was seen. (FIG. 7A). One micromolar of D-Bluco gave rise to a 37.6-fold increase in fluorescence (λ_{max excitation}=330 nm, λ_{max emission}=530 nm), but little emission across these wavelengths in the absence of TEM-1 (FIG. 7B).

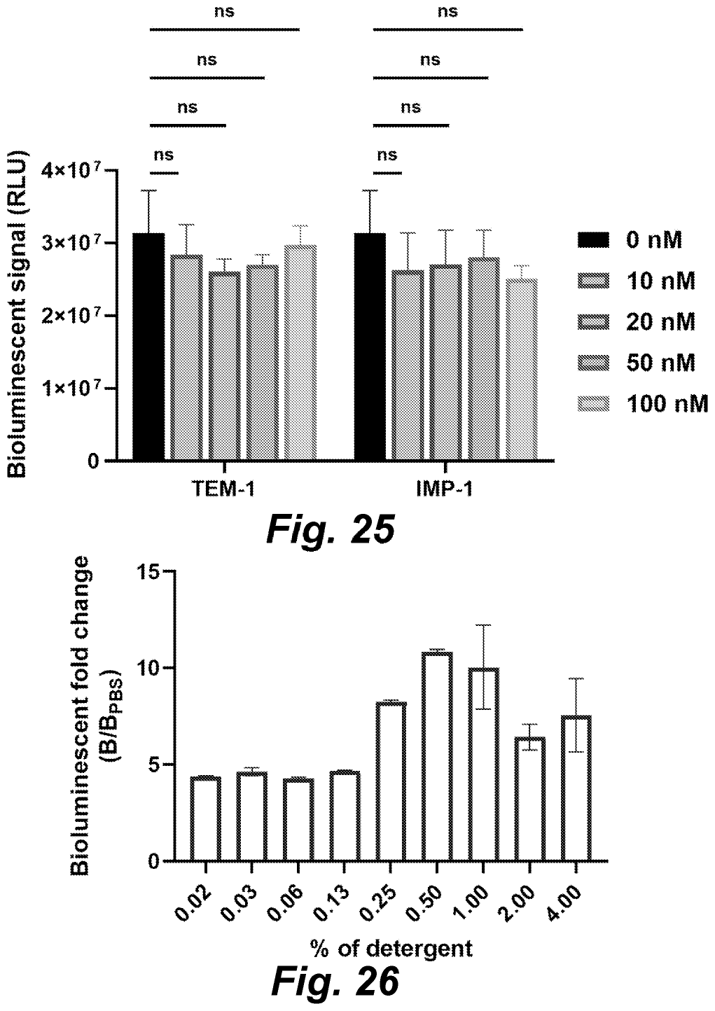

D-Bluco was highly stable when incubated with Escherichia coli (E. coli) or PBS. The bioluminescence emission from the released D-luciferin was measured. The bioluminescence emission was dependent on the incubation time with TEM-1 and the concentration of D-Bluco. Also evaluated was whether there was interference between luciferase and β-lactamases in such dual enzyme reaction system. No significant differences were noticed among the groups of D-luciferin with or without TEM-1 or IMP-1, a class B carbapenemase (FIG. 25). Importantly, as shown in FIG. 7C, D-Bluco produced a 1420-fold increase in bioluminescence emission upon TEM-1 treatment. Its initial bioluminescent signal was significantly lower than that of Bluco that only generated a 41.3-fold increase in the bioluminescence emission after 15 min TEM-1 treatment.

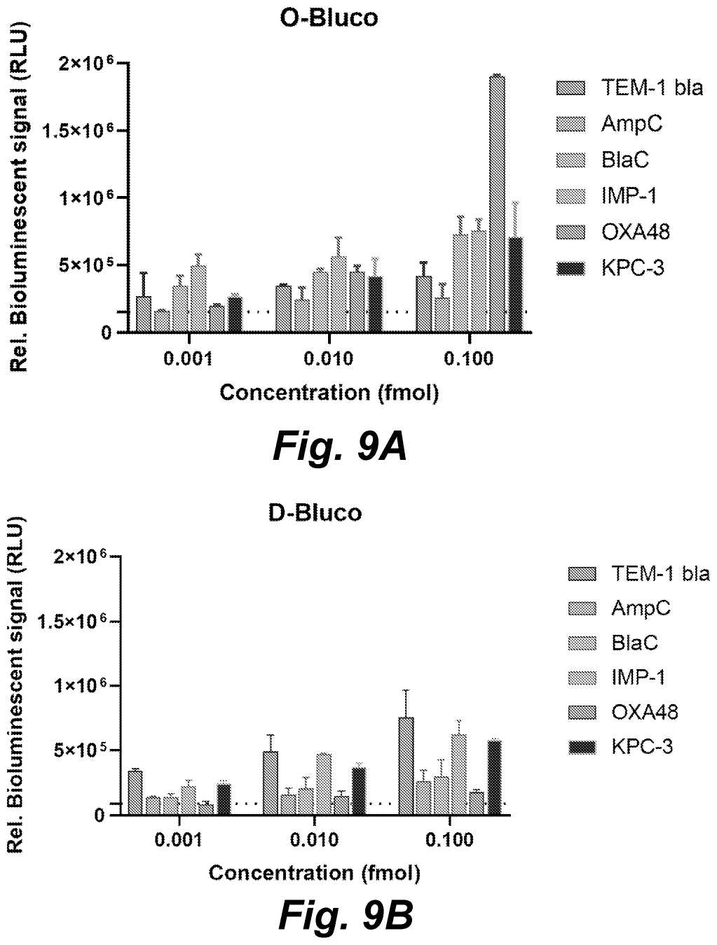

The initial bioluminescent background signal may come from residual D-luciferin present in the sample. High-performance liquid chromatography (HPLC) analysis could not detect any D-luciferin at a concentration below 0.1 μM. However, addition of firefly luciferase can efficiently detect and consume the residual free D-luciferin. There was still bioluminescence emission when additional batches of firefly luciferase (t₂ -t₄) were added to the Bluco solution (FIG. 9B), suggesting the existence of bioluminescence emission from other sources than from residual D-luciferin. In comparison, for D-Bluco, no further bioluminescence emission was detected besides the first flash (t₀) from the initial firefly luciferase addition.

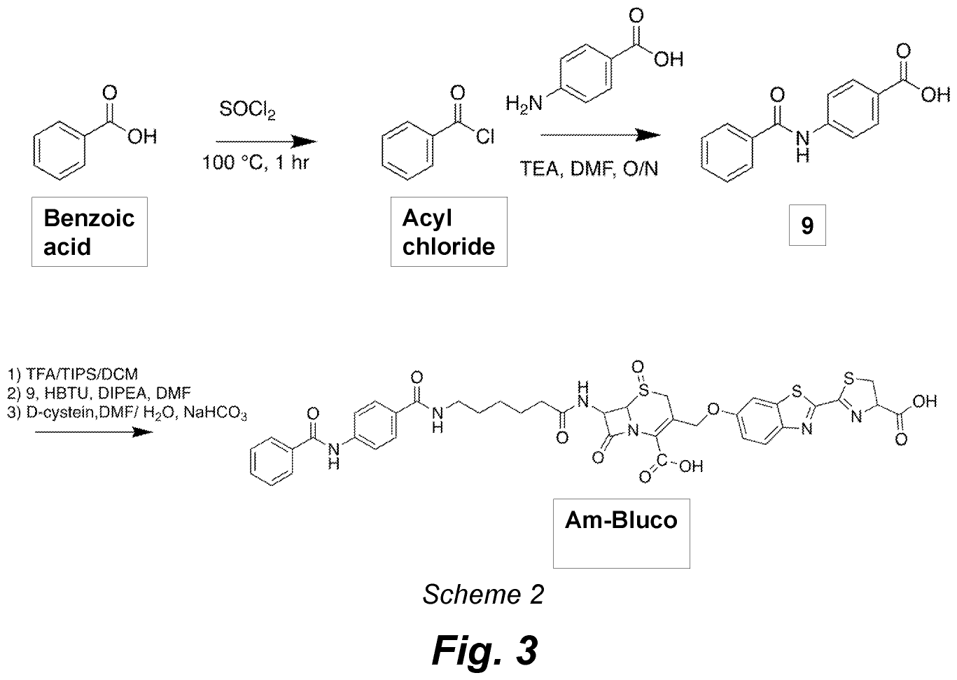

To further confirm the quenching effect of dabcyl in D-Bluco, another control, Am-Bluco, was prepared that is structurally similar to D-Bluco but with dabcyl replaced by a structure lacking the broad absorption between 400 and 530 nm (FIG. 1). Like Bluco, Am-Bluco produced bioluminescence emission upon each addition of firefly luciferase (FIG. 3). Mass spectrometry revealed that pro-luciferin probes Bluco, Am-Bluco, and D-Bluco were oxidized when incubated with firefly luciferase. These results together have established that caged pro-luciferin probes can produce background bioluminescence emission before uncaging and that the attached dabcyl group can quench this background emission via bioluminescence resonance energy transfer (BRET).

Having confirmed the quenching ability of dabcyl to lower D-Bluco bioluminescent background emission, the activity of D-Bluco towards a variety of β-lactamases that are of clinical relevance in addition to TEM-1, including KPC-3, BlaC (Class A), AmpC (Class C), and OXA-48 (Class D) was then assessed. These enzymes were recombinantly expressed and purified. The bioluminescent signal was measured with an IVIS optical imaging system after treating D-Bluco with each enzyme for 2 hours at room temperature. All these β-lactamases triggered significant bioluminescence in hydrolyzing Bluco and D-Bluco (FIG. 9B).

Carbapenemase OXA-48 is more sensitive to large substitution near the hydrolytic site and shows a low efficiency in hydrolyzing D-Bluco. The limit of detection (LOD) of D-Bluco for these β-lactamases was quantified by calculating the bioluminescent signal of three times of the standard deviation of the negative controls (D-Bluco in PBS). D-Bluco could detect OXA-48 and AmpC at 0.1 femtomole while all other β-lactamases at the quantity of as low as 0.001 femtomole. This sensitivity reflects a 10-100 fold improvement over previously reported fluorescent cephalosporin probes.

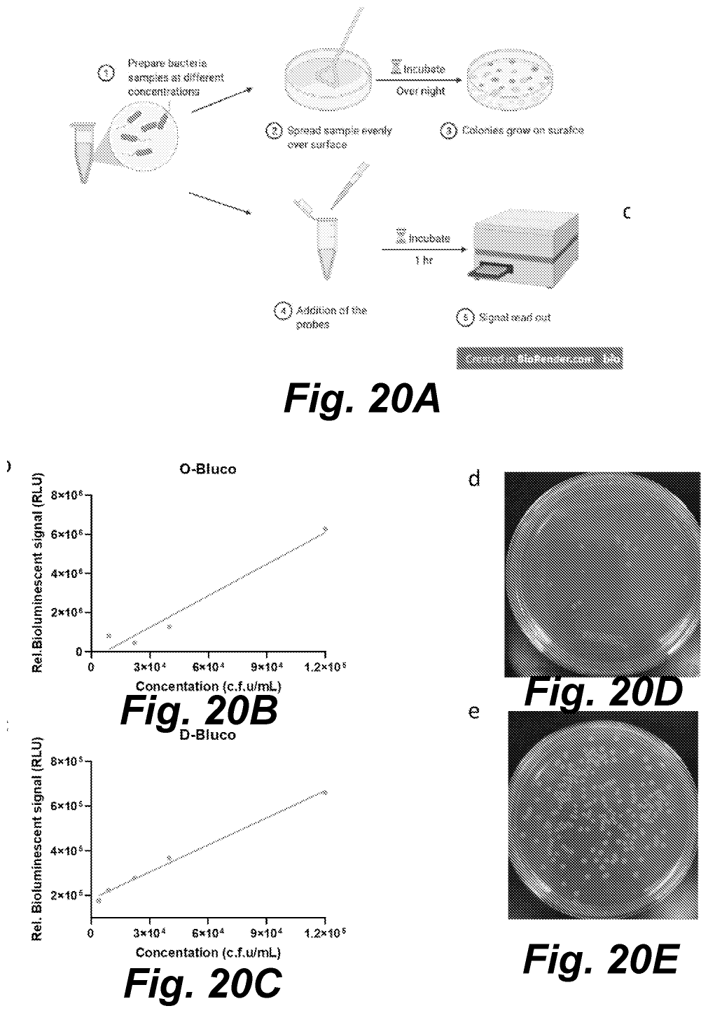

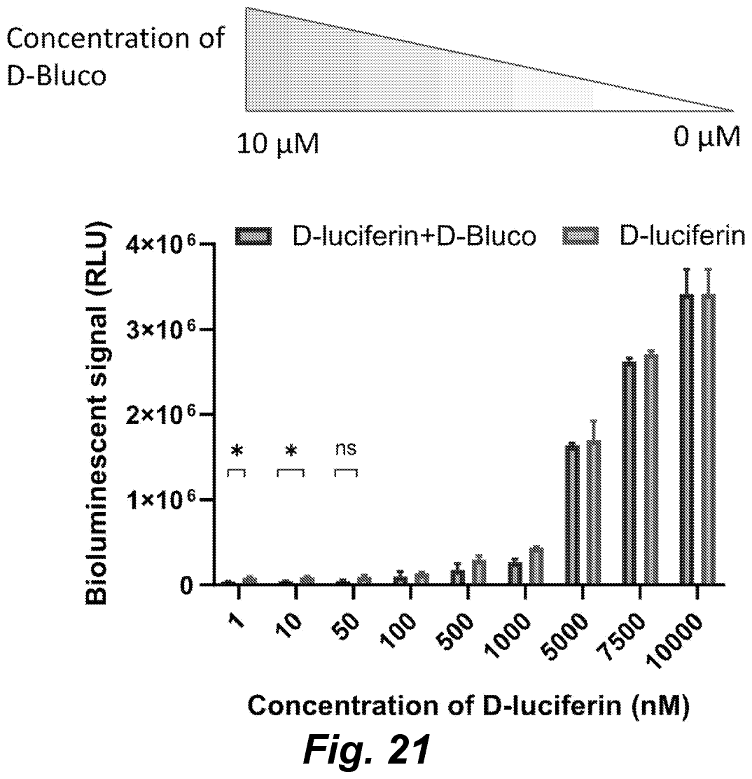

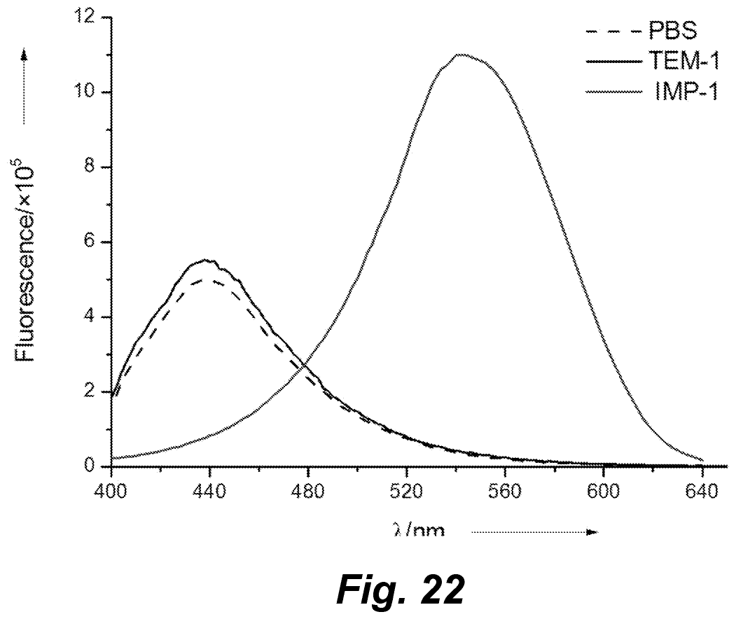

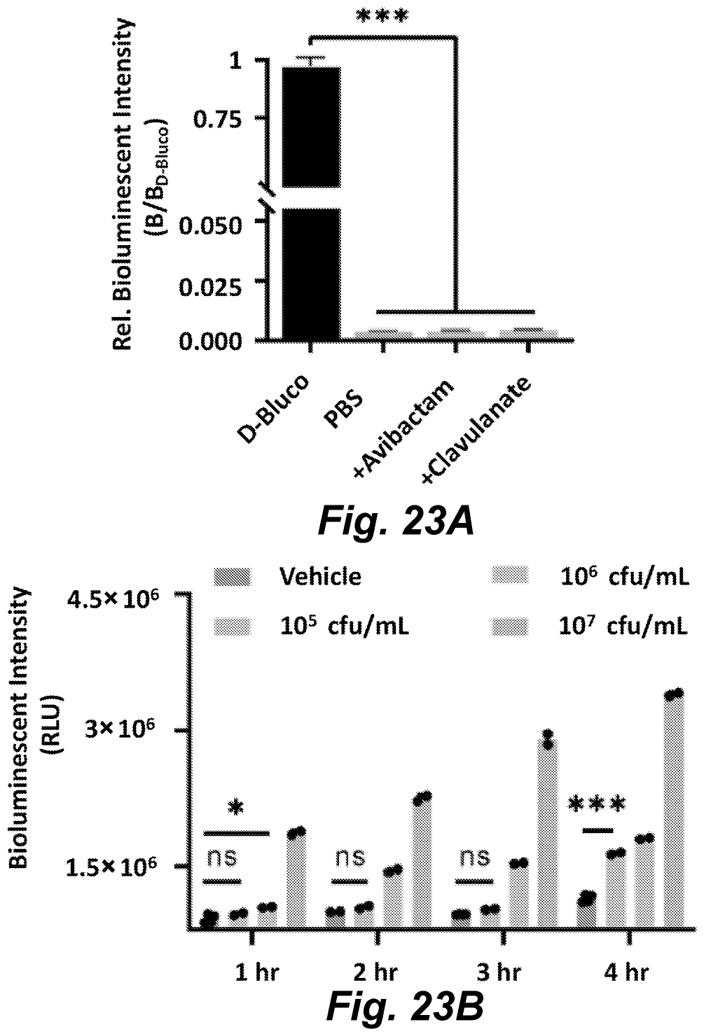

Transformed E. coli expressing TEM-1 (E. coli/TEM-1) was evaluated with D-Bluco. Parental E. coli (BL21) was used as a negative control. The number of bacteria present in the assay was validated by plating method (FIGS. 20A and 20C). After an hour incubation of D-Bluco with E. coli/TEM-1, a positive correlation between the bioluminescent signal and concentration of bacteria (cfu/mL) was observed, and as low as 102 cfu/mL of bacteria was detected within an hour—10 cfu in a 100 μL volume. In contrast, a 20 hr-incubation was required to detect 10³ cfu/mL TEM-1 expressing bacteria with a recently reported 3,7-diester phenoxazine probe, CDA, (Cephalosporin caged Diester Amplex red analogue). The detection specificity of D-Bluco was confirmed by including β-lactamase inhibitors avibactam and clavulanate in the incubation. The bioluminescence signals were abolished in the presence of inhibitors.

A clinically isolated E. coli strain producing TEM-type β-lactamase (E. coli/TEM) was further tested. The bioluminescent signal increased in a time- and probe concentration-dependent manner. However, clinically isolated E. coli/TEM was detected at a concentration of 10⁶ cfu/mL after an hour incubation and 10⁵ cfu/mL within 4 hours incubation with D-Bluco. In comparison to the transformed strain expressing recombinant β-lactamase, the clinical isolate had much less copies of plasmids, and the degree of gene amplification within plasmids and the promoter efficiency could also vary, resulting in significant difference in the level of β-lactamases expressed.

To evaluate whether releasing β-lactamases from periplasm could improve detection sensitivity, the bacteria were lysed. 3-[(3-cho-I-amidopropyl)-dimethylammonio]-1-propanesul-fonate (CHAPS) is a non-denaturing zwitterionic detergent and has been used for bacterial lysis. 0.5% CHAPS significantly enhanced the bioluminescence signal in 10⁶ cfu/mL E. coli/TEM without a negative impact on the luciferase activity (FIG. 26).

In addition to E. coli, Klebsiella pneumoniae (K. pneumoniae) is among the most common nosocomial Enterobacterales capable of developing lactam-resistance. Two clinically significant isolates K. pneumoniae expressing KPC carbapenemase (K. pneumoniae/KPC) and E. coli expressing New Delhi metallo β-lactamase (E. coli/NDM) were evaluated.

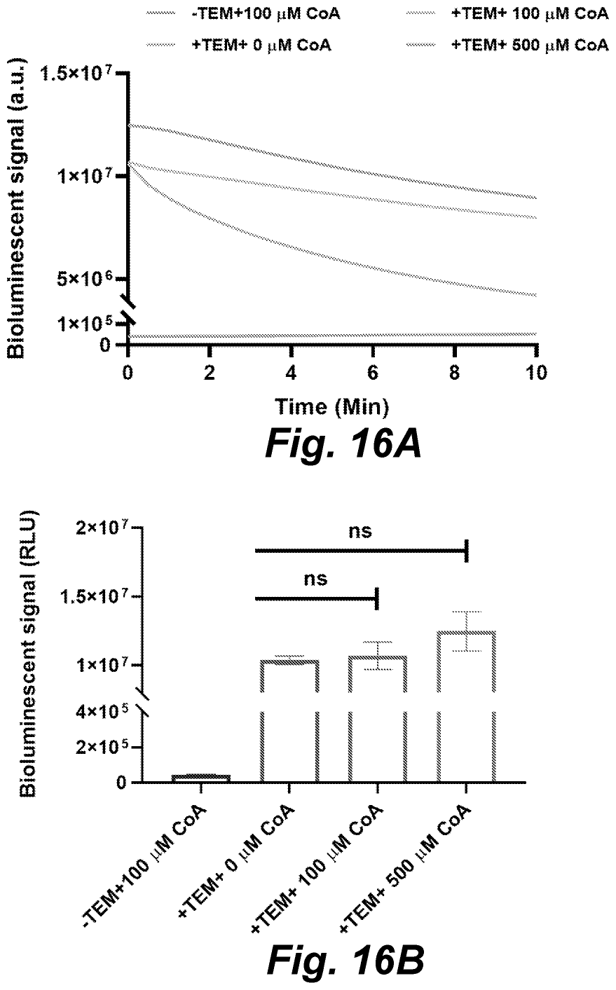

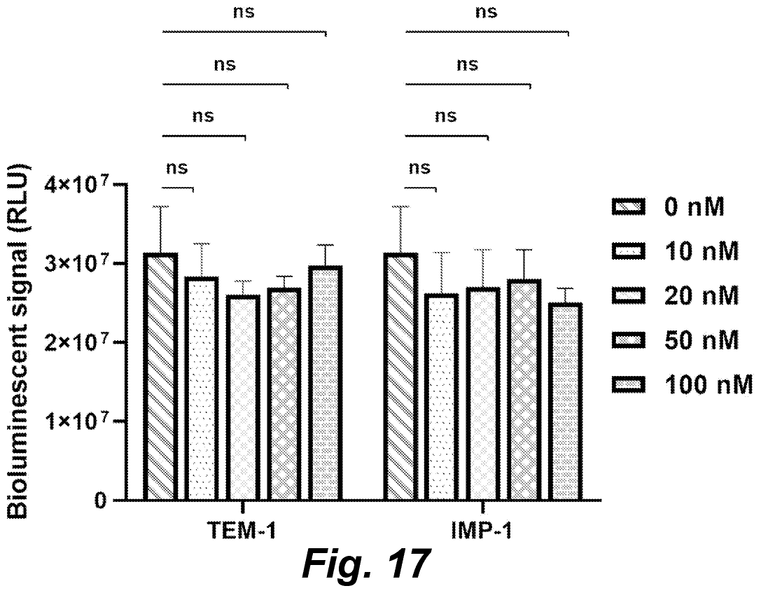

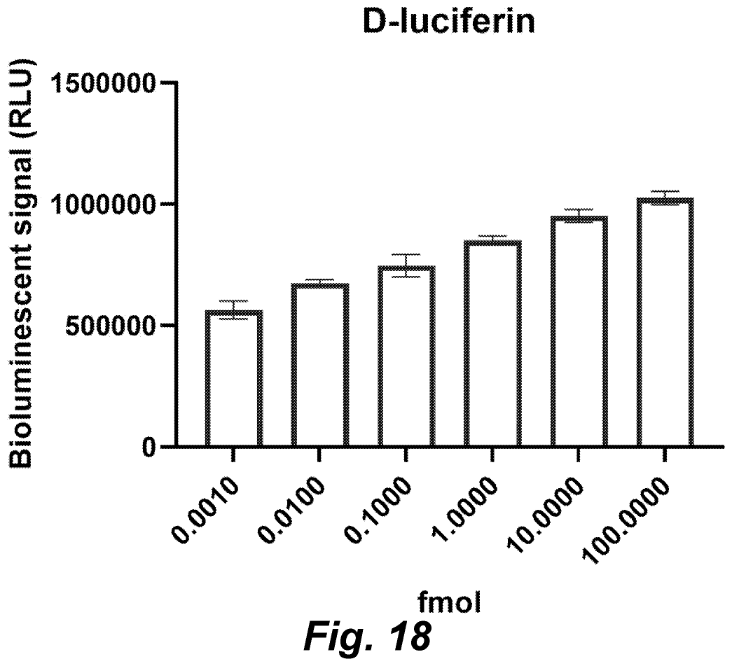

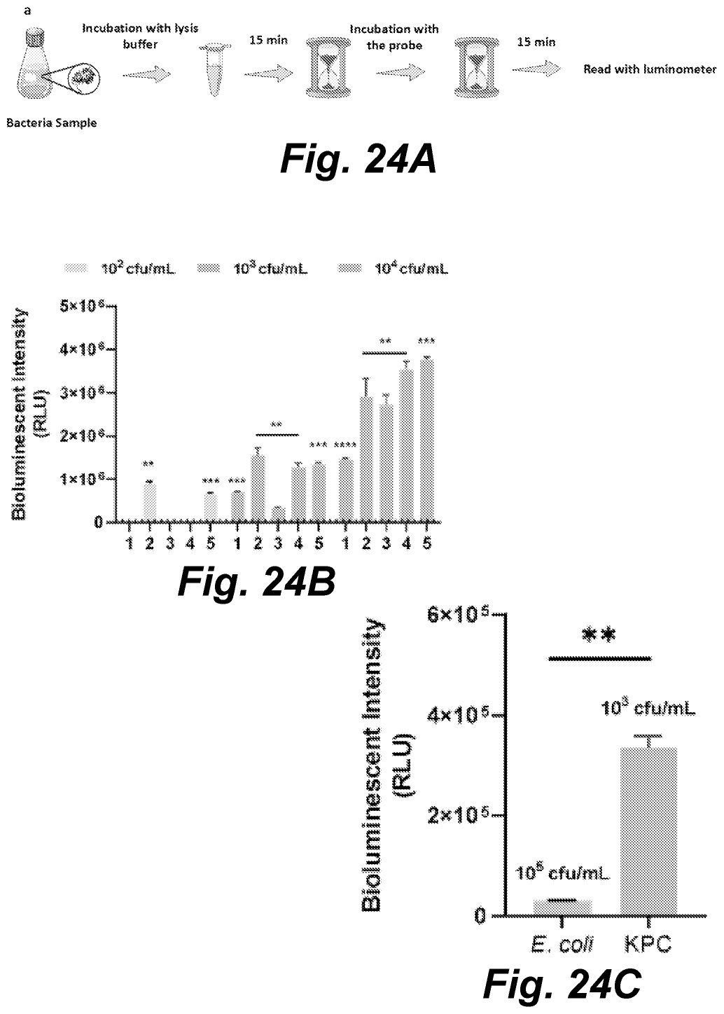

Compared to intact bacteria, incubating D-Bluco with E. coli/TEM, K. pneumoniae/KPC, or E. coli/NDM lysate generated 52-fold, 141-fold and 145-fold signal enhancement, respectively (FIG. 15). Moreover, to further improve the sensitivity of our assay, the assay volume was increased to 1 mL, a 10-fold increment, and attempted to introduce coenzyme A (CoA) to the assay. CoA has been suggested to help enhance bioluminescent signal by conducting a thiolytic reaction to block the inhibitory side product, dehydroluciferyl adenylate. While a stabilized signal was observed, no significant bioluminescent signal enhancement was achieved (FIGS. 16A and 16B). These results led to developing an optimized assay for rapid, extremely sensitive detection of β-lactamase activity, as shown in FIG. 24A. Bacteria was mixed with 0.5% CHAPS lysis buffer for 15 min, and then incubated with D-Bluco at room temperature for another 15 min. The resulted mixture was combined with assay buffer which contains MgCl₂, CoA, ATP, and luciferase for immediate bioluminescent reading.

Urinary tract infection (UTI) is one of the most common infections, affecting almost 50% of the population at least once in their life time and one of the largest groups for routine antibiotic administration. Overuse of antibiotics in UTI has raised a major concern in developing resistance. Many broad-spectrum antimicrobials are prescribed before an antibiotic susceptibility test (AST) report is available, especially in rapidly progressing infections. Subsequently, as many as 40% of patients may expose to unnecessary or inappropriate antibiotics. These broad-spectrum antibiotics can adversely affect the natural gut microbiota, thus exposing individuals to Clostridium difficile colitis and favoring resistance in specific bacteria strains. the clinical value of the RAPID BLI test for detecting β-lactamase expressing bacterial pathogens in UTI was assessed.

Clinic isolates representing different classes (class A: Enterobacter cloacae/IMI, E. coli/TEM, K. pneumoniae/KPC; class B: E. coli/NDM, and class C: E. cloacae/AmpC) were spiked into synthetic urine samples. As shown in FIGS. 24B and 24C, clinic isolates can be detected directly without culture at a concentration of 10² cfu/mL for E. coli/TEM and E. cloacae/AmpC and 10³ cfu/mL for all other bacteria. According to the UTI criteria defined by CDC (Centers for Disease Control and Prevention), a positive urine culture has ≥10⁵ cfu/mL of one but no more than two bacteria species. Therefore, the sensitivity of the RAPID BLI assay is way below this threshold. Compared to conventional methods, the assay of the present disclosure is both rapid and sensitive, and thus holds enormous potential to be used at the point-of-care.

The source of the bioluminescence background emission in bioluminogenic pro-luciferin probes was investigated. In addition to trace amounts of free D-luciferin in the sample, pro-luciferin probes can be oxidized by luciferases due to the promiscuity of the enzyme packet. It was demonstrated that suppressing this background by the attached broad spectrum quenching moiety via bioluminescence resonance energy transfer (BRET) mechanism could significantly improve the sensitivity. By combining BRET quenching and chemical caging, an ultrasensitive bioluminescent probe D-Bluco was developed that could detect as low as 10-18 moles of β-lactamase activity. It was demonstrated that a D-Bluco based bioluminescence assay (RAPID BLI) can detect β-lactamase activity in clinically bacteria isolates in urine samples in 30 mins with a superior sensitivity (10²-10³ cfu/mL).

The strategy of combining BRET quenching and chemical caging to suppress background and achieve high sensitivity can be an advantageous approach for the development of other bioluminescent sensors.

### β-Lactamase Response towards D-Bluco and O-Bluco

Both probes were evaluated to with TEM-1 bla, one of the most abundant β-Lactamases for initial testing. The fluorescent signal of D-Bluco and O-Bluco incubated with 100 nM TEM-1 bla was examined with a microplate reader over a period of 40 minutes. The fluorescent intensity of these caged luciferin derivatives increased only in the presence of the enzymes and enhanced with increasing compounds concentration (FIGS. 7A-7C). The fluorescence intensity for D-Bluco rapidly increased within 10 minutes of the reaction with TEM-1 bla, followed by a plateau, while O-Bluco showed a gradual increase of signal. In the presence of TEM-1 bla, D-Bluco gave a 37.6-fold increase in relative fluorescence emission attributed to the product luciferin [λₘₐₓ (excitation=330 nm), λₘₐₓ (emission=530 nm)], and O-Bluco gave a 32.7-fold increase in relative fluorescence emission (FIG. 7C).

The signal to background ratio for D-Bluco, O-Bluco, and Bluco was measured following the addition of the firefly luciferase (fLuc). While all three caged luciferin analogues revealed strong light emissions, D-Bluco gave the lowest background signal and therefore the highest turn-on ratio of 1420-fold within 15 min (bioluminescent signal with TEM-1 over the signal without TEM-1), an enhancement significantly higher than Bluco (41.3-fold) and O-Bluco (100.5-fold) (FIG. 8).

Enzyme CoA has been reported to help enhance bioluminescent signal of luciferase assay.²⁴ We thus tested its use in enhancing the bioluminescence signal. A slower decay of signal was observed but little bioluminescent signal enhancement was achieved (FIGS. 16A and 16B). Luciferase enzyme not impacting the activity of β-lactamases was verified.

D-Bluco and O-Bluco were evaluated with various β-lactamase that of clinical relevance (FIG. 9B and 9C), including KPC-3, BlaC, TEM-1 (Class A), IMP-1 (Class B), AmpC (Class C), and OXA-48 (Class D). These enzymes were recombinantly expressed, purified as previously described²⁵. The bioluminescent signal was measured after incubating O-Bluco or D-Bluco with each enzyme at different concentrations for 2 hr at r.t. The detection limits for the panel of enzymes are all within the femtomole range. This compares favorably to previously reported fluorescent cephalosporin probe with a 1000-fold sensitivity improvement.²⁶

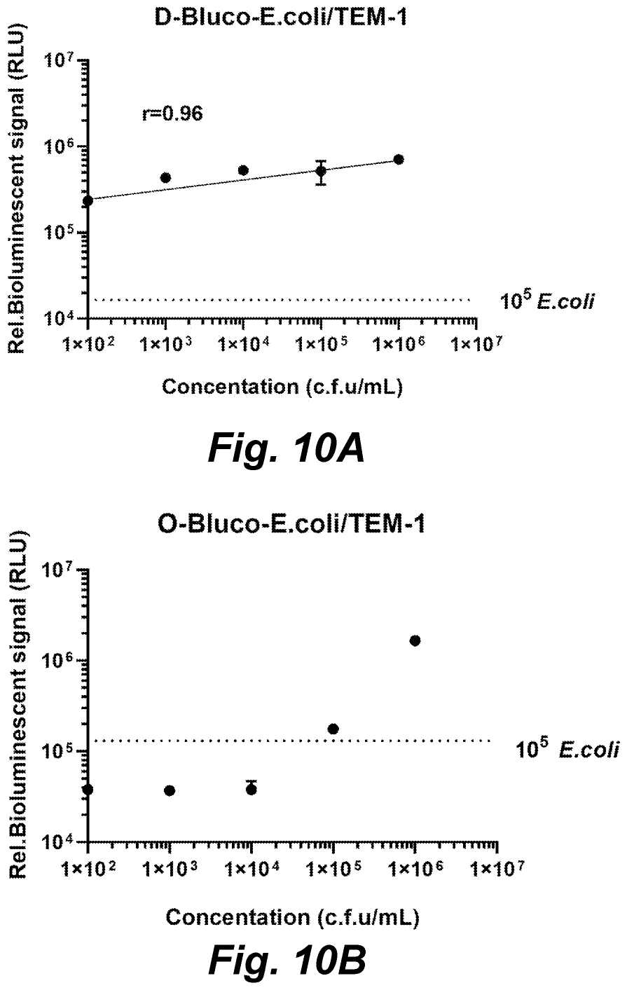

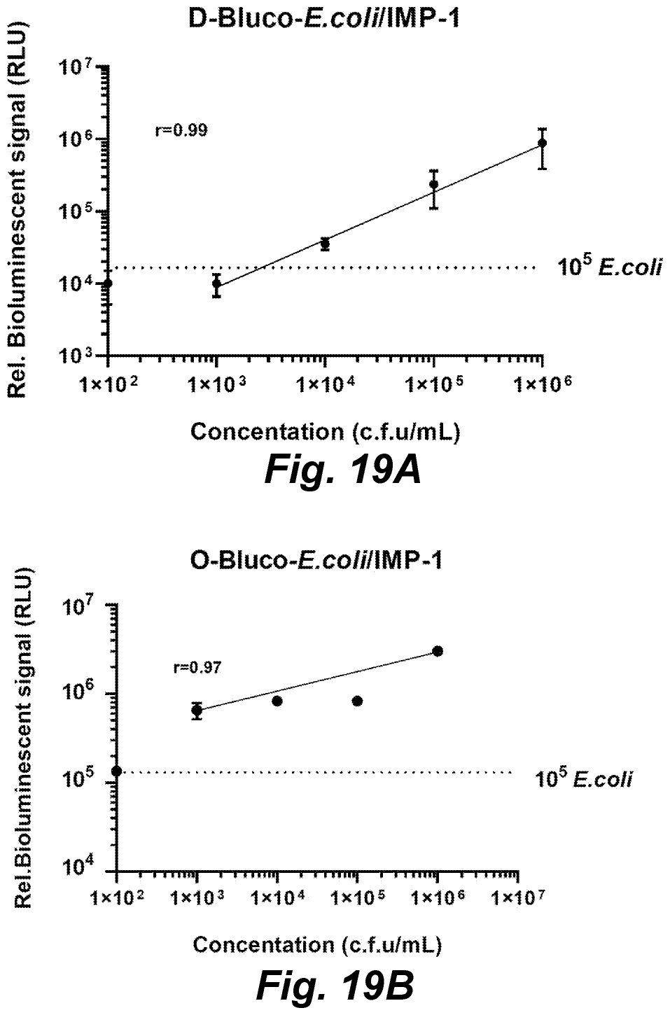

Rapid test assays with engineered bacteria. The D-Bluco and O-Bluco was incubated with different concentrations of TEM-1 and IMP-1 expressing Escherichia coli (E. coli) for an hour (FIGS. 10A, 10B, 19A, and 19B). E. coli was employed as a negative control. There was a positive correlation between the bioluminescent signal and bacteria concentration measured by colony forming unit (c.f.u/mL) observed for both D-Bluco and O-Bluco. The lowest number of E. coli/TEM-1 detected by D-Bluco was 10² c.f.u/mL, and 10⁵ c.f.u/mL for O-Bluco. The lowest concentration of IMP-1 expressing bacteria detected by D-Bluco was higher (10⁴ c.f.u/mL).

### Rapid Test Assays with Clinical Isolates

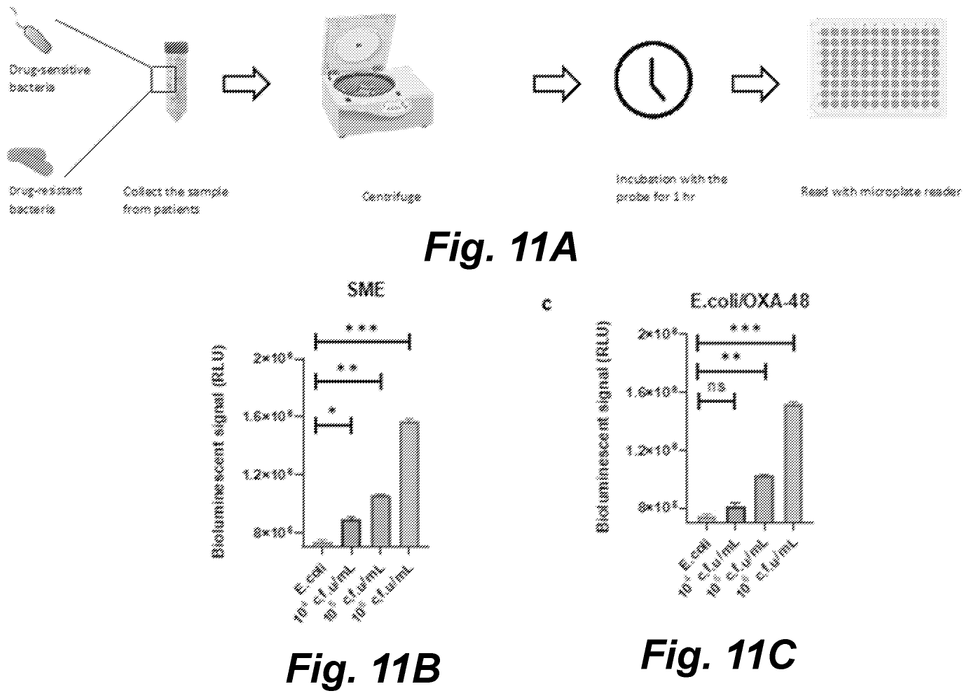

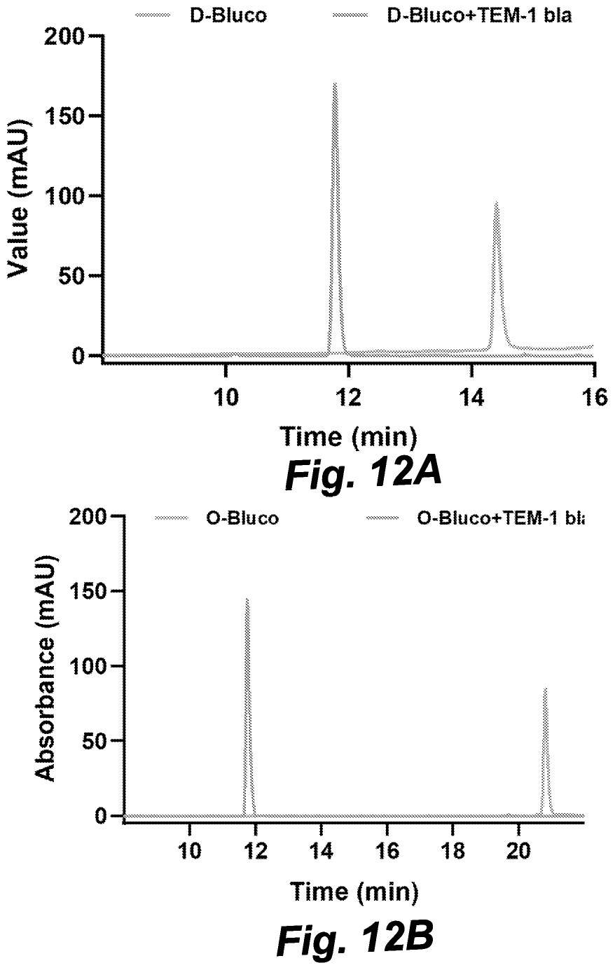

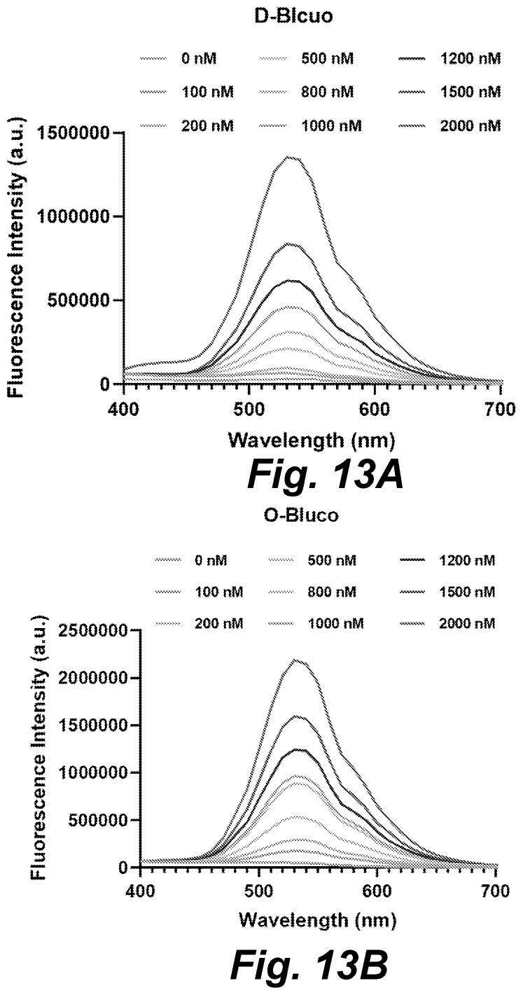

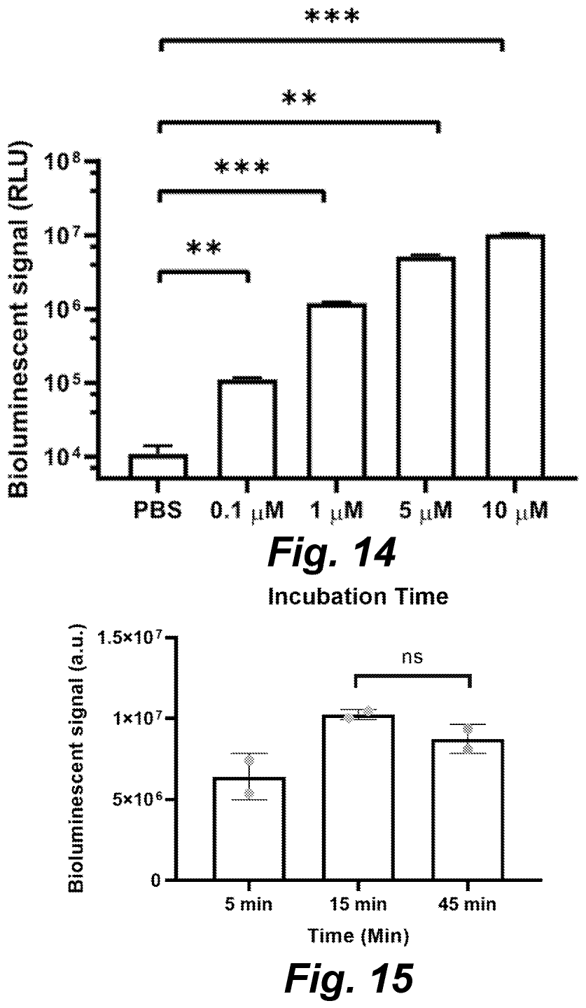

Urinary tract infection (UTI) is a severe public health problem because of the high recurrence rates and increasing antimicrobial resistance. It is among the major cause of patient morbidity and health care expenditures for both men and women of all age groups, accounting for 1 million cases of nosocomial in USA annually and an estimate cost of 1.6 billion. As a proof-of-concept, we spiked different concentrations of clinic isolates into synthetic urine samples. E. coli expressing OXA-48 and S. marcescens (SME) were chosen as examples. S. marcescens, being an opportunistic pathogen, accounts for an average of 6.5% of all Gram negative infection in intensive Care Units. The spiked urine samples were filtered and centrifuged to collect the bacteria (FIG. 11A). The collected bacteria were then diluted in PBS (pH=7.4) and incubated with 10 μM D-Bluco for an hour at 37° C. D-Bluco provided significant bioluminescence enhancement for SME starting at 10⁴ c.f.u/mL and for E. coli expressing OXA-48 starting at 10⁵ c.f.u/mL within an hour (FIGS. 11B and 11C).

### Synthesis of CP-Luc and CPL-luc for Carbapenemases Detection

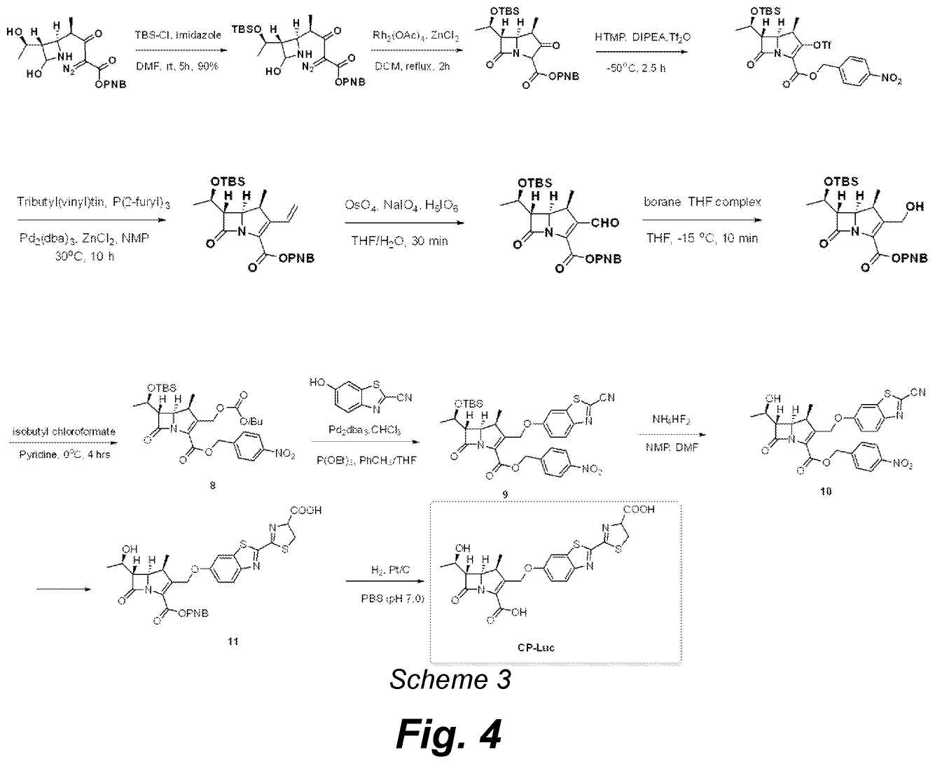

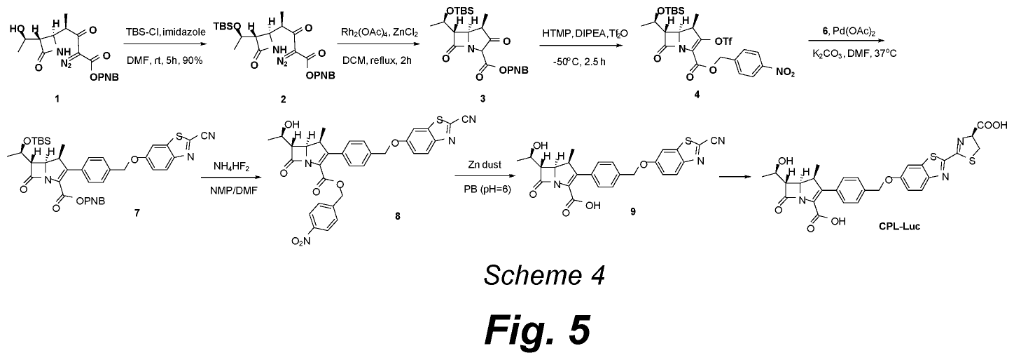

In addition, the cephalosporin moiety in O-Bluco was replaced with a carbapenem to afford CP-Luc (Scheme 3, FIG. 4) and CPL-Luc (Scheme 4, FIG. 5) for the specific detection of carbapenemase enzymes.

One aspect of the present disclosure encompasses embodiments of a caged bioluminescent probe comprising a luciferin moiety, an enzyme-cleavable moiety, and a caging moiety, wherein the moieties are conjugated to form the structure having the formula:

(luciferin moiety)-(enzyme-cleavable moiety)-(caging moiety),

and wherein the enzyme-cleavable moiety can be cleaved by a bacterial enzyme.

In some embodiments of this aspect of the disclosure, the caged bioluminescent probe can further comprise a linker moiety between the luciferin moiety and the enzyme-cleavable moiety.

In some embodiments of this aspect of the disclosure, the enzyme-cleavable moiety is cleavable by a β-lactamase.

In some embodiments of this aspect of the disclosure, the caging moiety can be a quencher.

In some embodiments of this aspect of the disclosure, the quencher can be a diabcyl quencher.

In some embodiments of this aspect of the disclosure, the caged bioluminescent probe can have the formula:

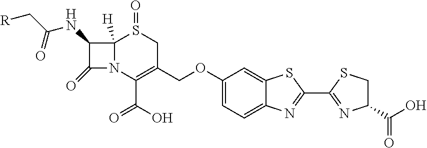

wherein R is

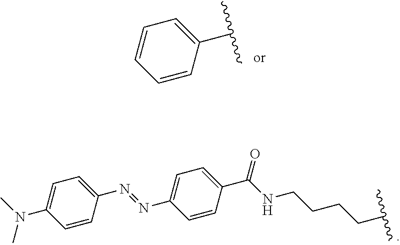 [SMILES: *c1ccccc1 ; *CCCCNC(=O)c1ccc(/N=N/c2ccc(N(C)C)cc2)cc1]

In some embodiments of this aspect of the disclosure, the enzyme-cleavable moiety can be cleavable by a carbapenemase.

In some embodiments of this aspect of the disclosure, the caged bioluminescent probe can have the formula:

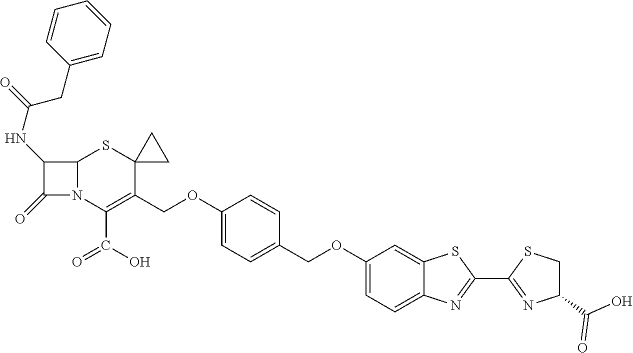 [SMILES: O=C(Cc1ccccc1)NC1C(=O)N2C(C(=O)O)=C(COc3ccc(COc4ccc5nc(C6=N[C@@H](C(=O)O)CS6)sc5c4)cc3)C3(CC3)SC12]

wherein the linker has the structure

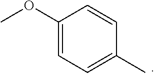

In some embodiments of this aspect of the disclosure, the caged bioluminescent probe can have the formula:

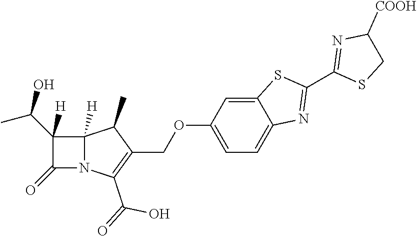 [SMILES: C[C@@H](O)[C@H]1C(=O)N2C(C(=O)O)=C(COc3ccc4nc(C5=NC(C(=O)O)CS5)sc4c3)[C@H](C)[C@H]12]

In some embodiments of this aspect of the disclosure, the caged bioluminescent probe can further comprise a linker moiety between the luciferin moiety and the enzyme-cleavable moiety

In some embodiments of this aspect of the disclosure, the caged bioluminescent probe can have the formula:

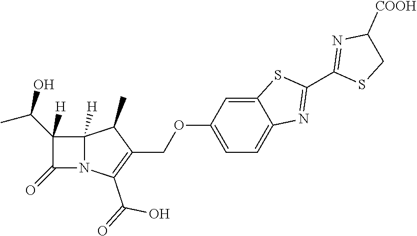 [SMILES: C[C@@H](O)[C@H]1C(=O)N2C(C(=O)O)=C(COc3ccc4nc(C5=NC(C(=O)O)CS5)sc4c3)[C@H](C)[C@H]12]

wherein the linker has the structure

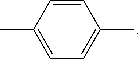

Another aspect of the present disclosure encompasses embodiments of a method of identifying a bacterial strain resistant to a β-lactam antibiotic or a carbapenem antibiotic, wherein said method can comprise contacting a population of bacteria with a caged bioluminescent probe cleavable by either a β-lactamase or a carbapenemase, adding luciferinase; and measuring an emitted bioluminescent signal, wherein a detected emitted bioluminescent signal indicates that the bacterial strain has a β-lactamase or a carbapenemase activity.

In some embodiments of this aspect of the disclosure, the caged bioluminescent probe can comprise a luciferin moiety, an enzyme-cleavable moiety, and a caging moiety, wherein the moieties are conjugated to form the structure having the formula:

(luciferin moiety)-(enzyme-cleavable moiety)-(caging moiety),

and wherein the enzyme-cleavable moiety can be cleaved by a bacterial enzyme.

In some embodiments of this aspect of the disclosure, the caged bioluminescent probe can comprise a linker moiety between the luciferin moiety and the enzyme-cleavable moiety.

In some embodiments of this aspect of the disclosure, the enzyme-cleavable moiety is cleavable by a β-lactamase or by a carbapenemase.

In some embodiments of this aspect of the disclosure, the caging moiety can be a quencher.

In some embodiments of this aspect of the disclosure, the caged bioluminescent probe can have the formula:

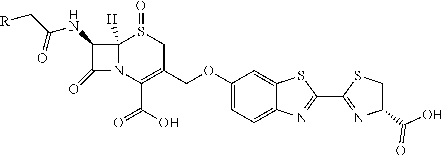

wherein R is

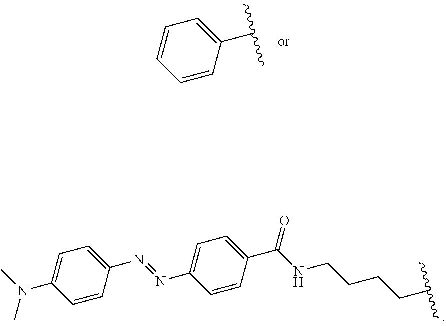 [SMILES: *c1ccccc1 ; *CCCCNC(=O)c1ccc(/N=N/c2ccc(N(C)C)cc2)cc1]

In some embodiments of this aspect of the disclosure, the caged bioluminescent probe can have the formula:

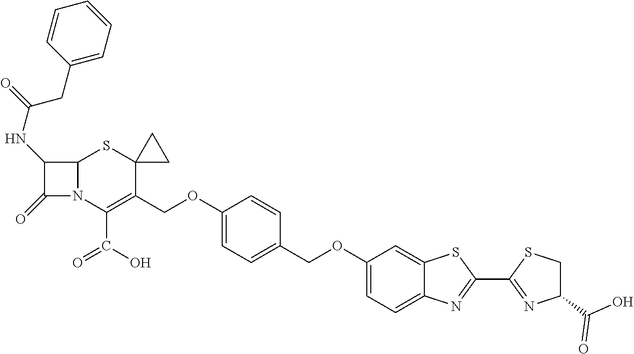 [SMILES: O=C(Cc1ccccc1)NC1C(=O)N2C(C(=O)O)=C(COc3ccc(COc4ccc5nc(C6=N[C@@H](C(=O)O)CS6)sc5c4)cc3)C3(CC3)SC12]

wherein the linker has the structure

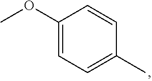

and wherein the enzyme-cleavable moiety can be cleaved by a carbapenemase.

In some embodiments of this aspect of the disclosure, the caged bioluminescent probe can have the formula:

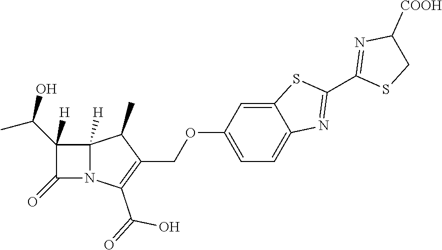 [SMILES: C[C@@H](O)[C@H]1C(=O)N2C(C(=O)O)=C(COc3ccc4nc(C5=NC(C(=O)O)CS5)sc4c3)[C@H](C)[C@H]12]

In some embodiments of this aspect of the disclosure, the caged bioluminescent probe can further comprise a linker moiety between the luciferin moiety and the enzyme-cleavable moiety.

In some embodiments of this aspect of the disclosure, the caged bioluminescent probe can have the formula:

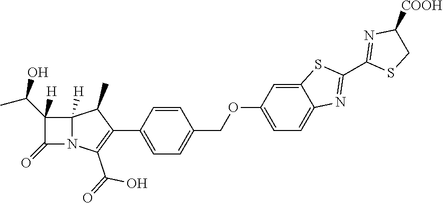 [SMILES: C[C@@H](O)[C@H]1C(=O)N2C(C(=O)O)=C(c3ccc(COc4ccc5nc(C6=N[C@@H](C(=O)O)CS6)sc5c4)cc3)[C@H](C)[C@H]12]

wherein the linker can have the structure

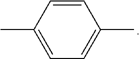

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

General information: All chemicals were purchased from commercial sources and used without further purification. The clone of TEM-1 Bla was described previously Fleming, A (Br. J. Exp. Pathol. 10: 226-236 (1929)). QuantiLum Recombinant luciferase (catalog number E1701) was purchased from Promega (Madison, WI). Reaction progress was monitored by analytical thin layer chromatography (TLC) with 0.25 mm silica gel 60F plates and visualized with fluorescent indicator (254 nm). Flash column chromatography was conducted using silica gel (SiliaFlash for flash column, 40-63 μm, 60 Å). The ¹H and ¹³C NMR spectra were acquired on a Varian 500 MHz or 600 MHz magnetic resonance spectrometer. Data for ¹H NMR spectra are reported as follows: chemical shifts are reported as δ in units of parts per million (ppm) relative to chloroform-d (δ 7.26, s); multiplicities are reported as follows: s (singlet), d (doublet), t (triplet), q (quartet), dd (doublet of doublets), m (multiplet), or br (broadened); coupling constants are reported as a J value in Hertz (Hz); and the number of protons (n) for a given resonance is indicated nH and based on the spectral integration values. High resolution mass spectra were obtained on a Thermo Exactive Orbitrap LC/MS. HPLC was performed on a Dionex HPLC System (Dionex Corporation) equipped with a GP50 gradient pump and an inline diode array UV-Vis detector. A reversed-phase C18 (Phenomenax, 5 μm, 10×250 mm or Dionex, 5 μm, 4.6×250 mm) column was used with a MeCN (B)/H₂O (A) gradient mobile phase containing 0.1% trifluoroacetic acid at a flow of 1 or 3 mL/min for the analysis. Fluorescence Spectra and kinetic experiments were collected by a SpectraMax iD3 multimode microplate reader (Molecular Device, San Jose, CA).

### Example 2

Bacteria growth and assay: E. coli (BL21) transformed to express TEM-1 Bla were grown in LB medium at 37° C. overnight and induced with 0.2% arabinose for 6-8 h at 30° C. 205 rpm. Colony forming units per milliliter (cfu/mL) were determined by measuring the UV absorbance at OD600. Clinically isolated K. pneumoniae expressing KPC, E. cloacae expressing IMI, E. coli expressing TEM, E. coli expressing NDM, and E. cloacae expressing AmpC were cultured in BD Columbia agar plate containing 5% sheep blood. Resistant bacteria colonies near meropenem discs were further inoculated in nutrient broth to culture overnight before use. For D-Bluco incubation, 10 μM working solution was prepared freshly by diluting stock solution (1 mM in pure DMSO) in PBS (pH 7.4).

### Example 3

Rapid test with engineered bacteria: E. coli expressing TEM-1 and E. coli expressing IMP-1 were cultured in Luria-Bertani (LB) broth at 37° C. overnight. Bacteria (OD600=1) were diluted to obtain different concentration (c.f.u/mL). To the bacteria solutions were added D-Bluco or O-Bluco (final concentration=10 M in PBS pH 7.4). The mixture was incubated for 1 h and the bioluminescent intensity in each entry was determined as described in the previous step. The c.f.u/mL of bacteria in each entry were further determined from the colonies on agar plate after serial dilutions.

### Example 4

Inhibitor test with recombinant bacteria: One hundred microliters (100 μL) of freshly cultured E. coli/TEM (OD600=0.5) were pretreated with PBS, 2 mg/mL potassium clavulanate or 2 mg/ml avibactam at 37° C. for 1 h. The bioluminescent signal was detected following the RAPID BLI test protocol.

### Example 5

Statistical analysis: GraphPad Prism 9 was used for plotting and statistical analysis. The statistical significance was calculated using the unpaired two-tailed Student's t test (** p<0.0021, **** p<0.0001).

### Example 6

### General Bioluminescent Measurement (Example as in FIGS. 3b and 3c).

The caged luciferin probes were incubated with different concentrations of TEM-1 β-lactamase or IMP-1 carbapenemase (0.001-0.1 fmol) in PBS (pH=7.4) for 2 hr at 25° C. Then a mixture of luciferase (200 nM), ATP (2 mM), and MgCl₂ (4 mM) was added, and bioluminescent signals were detected with a SpectraMax microplate reader or a luminator.

### Example 7

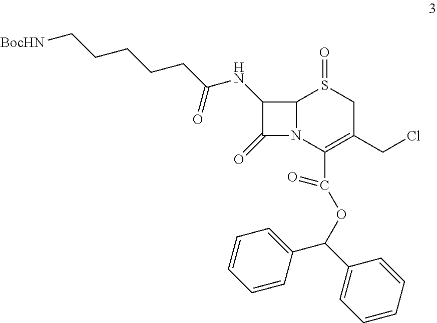 [SMILES: CC(C)(C)OC(=O)NCCCCCC(=O)NC1C(=O)N2C(C(=O)OC(c3ccccc3)c3ccccc3)=C(CCl)CS(=O)C12]

Benzhydryl 7-(6-((tert-butoxycarbonyl)amino)hexanamido)-3-(chloromethyl)-8-oxo-5-thia-1-azabicyclo[4.2.0]oct-2-ene-2-carboxylate (3): To a stirred suspension of PCl₅ (2.3g, 11.3 mmol) in DCM (5 mL) was added pyridine (0.89 g, 11.3 mmol) on ice. After 15 min, the solution was cooled to −40° C. and a solution of 3 (purchased from Pharmacore, China, 1.0 g, 1.88mmol) in DCM (10 mL) was added. The solution was subsequently stirred for 3 h before it was quenched by the addition of MeOH (2 mL) and mixture was stirred for another 30 min. The mixture was diluted with DCM (30 mL), washed with water, brine, dried over Na₂SO₄, filtered, and concentrated under reduced pressure to give a sticky brown solid. This solid was used for the next step without further purification. N-Boc-6-aminohexanoic Acid (278.74 mg, 1.2 mmol) in DMF (1 mL), was mixed with the crude product (500 mg, 0.113 mmol), and EDC (693.1 mg, 3.6 mmol). The mixture was stirred for 4 h before diluting it with ethyl acetate (50 mL) and washed with water (50 mL). The organic layer was separated, dried over Na₂SO₄, filtered, and concentrated under reduced pressure to give compound 2, which was used directly in the next step.

A solution of the crude compound 2 from previous step (60 mg, 0.096 mmol) in anhydrous DCM (5 mL) was cooled to 0° C. prior to the addition of meta-chloroperoxybenzoic acid (m-cpba, 21.9 mg, 1.0 mmol). The reaction was stirred at 0° C. for an hour. Silicon column purification afforded pure compound 3 (27.8 mg, 30.3% over 3 steps). ¹H NMR (600 MHz, DMSO): δ 8.49 (s, 1H), 8.16 (d, J=8.1 Hz, 1H), 8.11 (d, J=9.1 Hz, 1H), 7.95-7.93 (m, 2H), 7.86 (d, J=2.6 Hz, 1H), 7.77-7.74 (m, 3H), 7.27 (dd, J=9.1, 2.6 Hz, 1H), 6.80-6.78 (m, 2H), 5.79-5.71 (m, 1H), 5.11 (d, J=12.0 Hz, 1H), 4.89 (dd, J=4.8, 1.6 Hz, 1H), 4.83 (d, J=12.1 Hz, 1H), 3.92 (d. J=18.5 Hz, 1H), 3.61 (s, 1H), 3.24 (d, J=6.5 Hz, 2H), 3.03 (s, 9H), 2.31-2.24 (m, 1H), 2.23-2.17 (m, 1H), 1.52 (p, J=8.5, 7.2 Hz, 5H), 1.31 (q, J=7.2 Hz, 3H). ¹³C NMR (126 MHz, CDCl3) § 173.33, 164.49, 159.63, 156.04, 138.87, 138.78, 128.72, 128.59, 128.40, 128.31, 127.63, 127.00, 125.53, 121.32, 80.56, 79.14, 67.09, 59.00, 46.82, 43.55, 40.39, 36.08, 29.77, 28.47, 26.33, 24.94. LCMS: Calculated for C₃₂H₃₈ClN₃O₆S ([M+Na]⁺):667.18. Found:667.

### Example 8

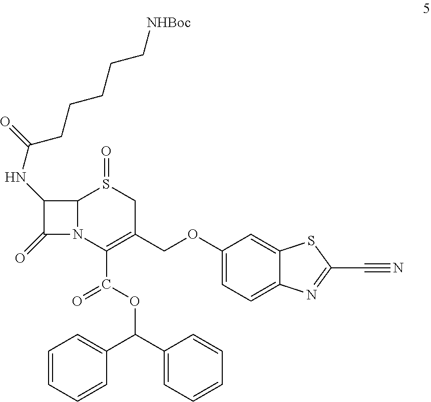 [SMILES: CC(C)(C)OC(=O)NCCCCCC(=O)NC1C(=O)N2C(C(=O)OC(c3ccccc3)c3ccccc3)=C(COc3ccc4nc(C#N)sc4c3)CS(=O)C12]

Benzhydryl 7-(6-((tert-butoxycarbonyl)amino) hexanamido)-3-(((2-cyanobenzo[d]thiazol-6-yl)oxy)methyl)-8-oxo-5-thia-1-azabicyclo[4.2.0]oct-2-ene-2-carboxylate 5-oxide (5). A mixture of the 3 (150 mg, 0.23 mmol) and sodium iodide (105 mg, 0.70 mmol) in 10 mL of acetone was stirred for 1.5 hour at ambient temperature. The reaction mixture was concentrated under reduced pressure and diluted with 5 ml of water. The suspension was extracted with ethyl acetate, and the organic phase was washed with 10% Na₂S₂O₃, H₂O, brine and dried over Na₂SO₄. The solvent was removed to get the title iodide 4, which is used for the next step without further purification.

A mixture of 6-hydroxybenzo[d]thiazole-2-carbonitrile (61.6 mg, 0.35 mmol), KCO₃ (48.3 mg, 0.35 mmol) and 4 in acetonitrile was stirred at room temperature for 9 h. DCM was added and washed with water. Flash chromatography purification on silica gel column afforded (33.8 mg of compound 5 (55% yield over two steps). ¹H NMR (500 MHz, CDCl₃) δ 7.93 (d, J=9.1 Hz, 1H), 7.38-7.35 (m, 2H), 7.28-7.18 (m, 9H), 7.04-6.98 (m, 2H), 6.85 (s, 1H), 6.51 (d, J=9.9 Hz, 1H), 6.06 (dd, J=9.9, 4.8 Hz, 1H), 5.20 (d, J=13.8 Hz, 1H), 4.73 (d, J=13.8 Hz, 1H), 4.44 (d, J=4.8 Hz, 1H), 4.02-3.96 (m, 1H), 3.29 (d. J=19.0 Hz, 1H), 3.00 (d, J=7.0 Hz, 2H), 2.18 (td, J=7.4, 2.8 Hz, 2H), 1.59-1.54 (m, 2H), 1.42-1.33 (m, 2H), 1.31 (s, 9H), 1.26 (q, J=7.7 Hz, 2H). ¹³C NMR (126 MHz, CDCl₃) δ 172.94, 164.36, 159.87, 158.07, 155.86, 147.27, 138.75, 138.68, 137.21, 134.15, 128.64, 128.55, 128.35, 128.26, 127.42, 126.85, 126.04, 124.59, 122.12, 118.34, 112.88, 103.84, 67.73, 66.81, 58.90, 45.35, 40.26, 36.07, 29.70, 29.59, 28.33, 26.21, 24.81. LCMS: Calculated for C₄₀H₄₁N₅O₈S₂ ([M+H]⁺):784.24; Found: 784.92

### Example 9

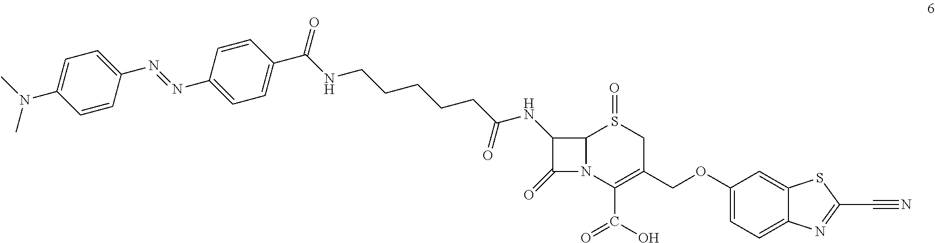 [SMILES: CN(C)c1ccc(/N=N/c2ccc(C(=O)NCCCCCC(=O)NC3C(=O)N4C(C(=O)O)=C(COc5ccc6nc(C#N)sc6c5)CS(=O)C34)cc2)cc1]

(E)-3-(((2-cyanobenzo[d]thiazol-6-yl)oxy)methyl)-7-(6-(4-((4-(dimethylamino)phenyl) diazenyl) benzamido)hexanamido)-8-oxo-5-thia-1-azabicyclo[4.2.0]oct-2-ene-2-carboxylic acid 5-oxide (6). To a solution of 5 (30 mg, 0.038 mmol) in 1.5 mL of dry dichloromethane was added TIPS (20 μL) and trifluoroacetic acid (200 μL) at 0° C. The mixture was stirred for 2 h at the same temperature, then the solvent was evaporated under reduced pressure and the residue was washed with ether (1 mL×3) and the product was used for the next step without further purification. This crude product was mixed with 2,5-dioxopyrrolidin-1-yl (E)-4-((4-(dimethylamino)phenyl)diazenyl)benzoate (9.8 mg, 0.027 mmol), triethylamine (TEA, 16 μL, 0.114 mmol) in DMF. The mixture was stirred at room temperature overnight. Silicon column purification afforded pure compound 6 (25.5 mg. 0.031 mmol, 80%). ¹H NMR (600 MHz, DMSO) δ 8.49 (s, 1H), 8.16 (d, J=8.1 Hz, 1H), 8.11 (d, J=9.1 Hz, 1H), 7.95-7.93 (m, 2H), 7.86 (d, J=2.6 Hz, 1H), 7.77-7.74 (m, 3H), 7.27 (dd, J=9.1, 2.6 Hz, 1H), 6.80-6.78 (m, 2H), 5.79-5.71 (m, 1H), 5.11 (d, J=12.0 Hz, 1H). 4.89 (dd, J=4.8, 1.6 Hz, 1H), 4.83 (d, J=12.1 Hz, 1H), 3.92 (d, J=18.5 Hz, 1H), 3.61 (s, 1H), 3.24 (d. J=6.5 Hz, 2H), 3.03 (s, 6H), 2.31-2.24 (m, 1H), 2.23-2.17 (m, 1H), 1.52 (p, J=8.5, 7.2 Hz, 4H), 1.30 (t, J=7.4 Hz, 2H). Calculated for C₃₇H₃₆N₈O₇S₂ [M-H]⁺): 767.21, LCMS Found: 767.4.

### Example 10

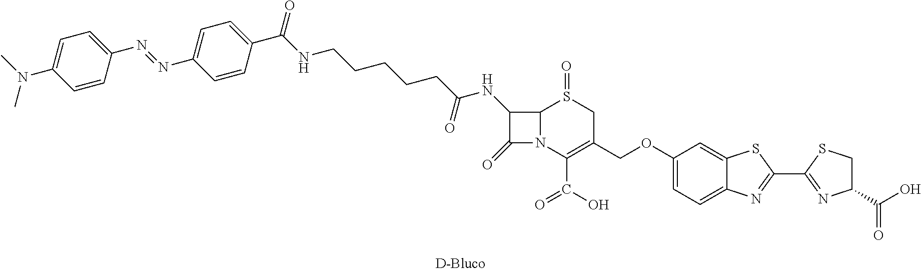 [SMILES: CN(C)c1ccc(/N=N/c2ccc(C(=O)NCCCCCC(=O)NC3C(=O)N4C(C(=O)O)=C(COc5ccc6nc(C7=N[C@@H](C(=O)O)CS7)sc6c5)CS(=O)C34)cc2)cc1]

3-(((2-((S)-4-carboxy-4,5-dihydrothiazol-2-yl)benzo[d]thiazol-6-yl)oxy)methyl)-7-(6-(4-((E)-(4-(dimethylamino)phenyl) diazenyl) benzamido)hexanamido)-8-oxo-5-thia-1-azabicyclo[4.2.0]oct-2-ene-2-carboxylic acid 5-oxide (D-Bluco). D-Bluco was prepared similarly as that of O-Bluco. Briefly, to a solution of compound 6 (10 mg, 0.013 mmol) in 1.5 mL of DMF was added a solution of D-cysteine hydrochloride (6.9 mg, 0.04 mmol) and NaHCO₃ solution (1 M) was added to adjust the pH>7 at room temperature. The reaction mixture was stirred for 20 min at room temperature. The product was purified with semi-prep HPLC to afford D-Bluco (3.4 mg, 30%). ¹H NMR (500 MHz, DMSO) δ 8.55 (t, J=5.6 Hz, 1H), 8.23 (d, J=8.1 Hz, 1H), 8.05 (d, J=9.0 Hz, 1H), 7.98 (d, J=8.5 Hz, 2H), 7.84-7.76 (m, 5H), 7.19 (dd, J=9.0, 2.6 Hz, 1H), 6.82 (d. J=9.1 Hz, 2H), 5.81 (dd, J=8.0, 4.7 Hz, 1H), 5.43 (dd, J=9.7, 8.3 Hz, 1H), 5.13 (d. J=11.9 Hz, 1H), 4.94 (d, J=4.4 Hz, 1H), 4.84 (d, J=11.9 Hz, 1H), 3.97 (s, 1H), 3.93 (s, 1H), 3.82-3.74 (m, 1H), 3.69 (d, J=8.4 Hz, 1H), 3.70-3.63 (m, 1H), 3.27 (d, J=6.4 Hz, 1H), 3.07 (s, 6H), 2.35-2.27 (m, 1H), 2.26-2.20 (m, 1H), 1.58-1.52 (m, 5H), 1.33 (s, 2H). ¹³C NMR (126 MHz, CDCl₃) δ 173.08, 164.49, 160.01, 158.21, 147.40, 138.88, 138.82, 137.34, 134.29, 128.77, 128.68, 128.48, 128.39, 127.56, 126.99, 126.18, 124.73, 122.25, 118.47, 113.02, 103.97, 80.59, 79.14, 67.87, 66.95, 59.04, 45.49, 40.39, 36.20, 29.83, 29.73, 28.46, 26.35, 24.94. HRMS: Calculated for C₄₀H₄₀N₈O₉S₃, [M+Na]⁺): 872.21; Found: 872.5570.

### Example 11

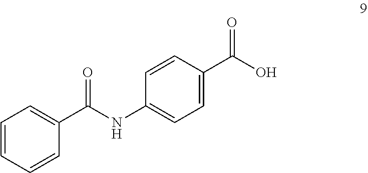 [SMILES: O=C(O)c1ccc(NC(=O)c2ccccc2)cc1]

4-benzamidobenzoic acid (9). The synthesis procedure was adopted from Dai et al., J. Am. Chem. Soc. (2020) 142: 15259-15264. Briefly, benzoic acid (1 g, 8.18 mmol) was added to thionyl chloride (1 mL) and DMF. The reaction mixture was refluxed at 100° C. for 1 h. The excess thionyl chloride and solvent was removed in vacuo. The acyl chloride obtained above was slowly added to the solution of 4-amino benzoic acid (1.34 g, 1.2 eq) and NEts (1.43 mL, 1.25 eq) in DCM (1 mL) at 0° C. The reaction mixture was stirred at room temperature overnight. The mixture was diluted with CH₂Cl₂ and washed with 3 M HCl aq. (2×30 mL), water (1×30 mL), and brine (1×30 mL). The organic layer was dried over MgSO₄, filtered, and concentrated under reduced pressure. Purification of the crude residue by flash column chromatography on silica gel afforded the corresponding benzamide substrates (505 mg, 45%). MS calculated for C₁₄H₁₁NO₃ ([M+H]⁺): 242.25; found: 242.13.

### Example 12

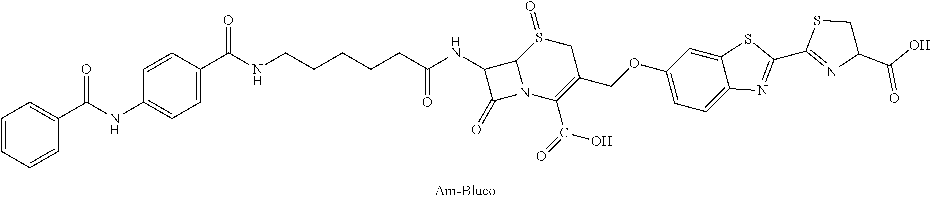 [SMILES: O=C(CCCCCNC(=O)c1ccc(NC(=O)c2ccccc2)cc1)NC1C(=O)N2C(C(=O)O)=C(COc3ccc4nc(C5=NC(C(=O)O)CS5)sc4c3)CS(=O)C12]

7-(6-(4-benzamidobenzamido) hexanamido)-3-(((2-(4-carboxy-4,5-dihydrothiazol-2-yl)benzo[d]thiazol-6-yl) oxy)methyl)-8-oxo-5-thia-1-azabicyclo[4.2.0]oct-2-ene-2-carboxylic acid 5-oxide (Am-Bluco): The synthesis is similar to that of compound 6 and D-Bluco (30%). ¹H NMR (600 MHz, DMSO) δ 10.41 (s, 1H), 8.34 (t, J=5.7 Hz, 1H), 8.18 (d, J=8.2 Hz, 1H), 8.05 (d. J=9.0 Hz, 1H), 7.96-7.94 (m, 3H), 7.87-7.82 (m, 3H), 7.78 (d, J=2.6 Hz, 1H), 7.61-7.57 (m, 1H), 7.52 (d, J=7.5 Hz, 2H), 7.19 (dd, J=9.0, 2.6 Hz, 1H), 5.81 (dd, J=8.1, 4.7 Hz, 1H), 5.42 (dd, J=9.8, 8.3 Hz, 1H), 5.14 (d, J=12.1 Hz, 1H), 4.94 (d, J=4.6 Hz, 1H), 4.86 (d, J=12.1 Hz, 1H), 3.99 (d, J=18.5 Hz, 1H), 3.77 (dd, J=11.2, 9.8 Hz, 1H), 3.71-3.65 (m, 2H), 3.24 (q, J=6.6 Hz, 2H), 2.27 (ddd, J=39.8, 14.4, 7.2 Hz, 2H), 1.54 (dd, J=14.5, 7.2 Hz, 5H), 1.33 (q, J=7.9 Hz, 3H). MS: calculated for C₃₉H₃N₆O₁₀S₃ ([M-H]⁻): 843.93; found: 843.235.

### Example 13

Synthesis of CP-Luc and CPL-luc for carbapenemase detection: In addition, the cephalosporin moiety in O-Bluco was replaced with a carbapenem to afford CP-Luc (Scheme 3, FIG. 4) and CPL-Luc (Scheme 4, FIG. 5) for the specific detection of carbapenemase enzymes.

### Example 14

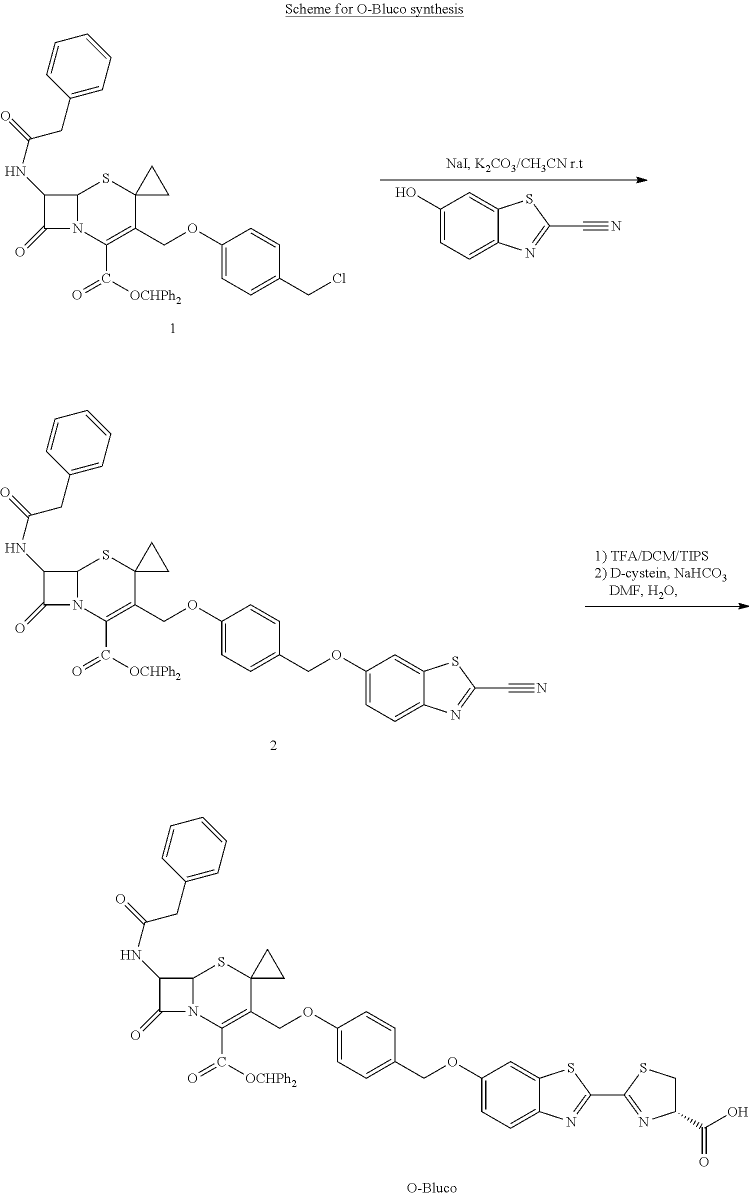

### Example 15

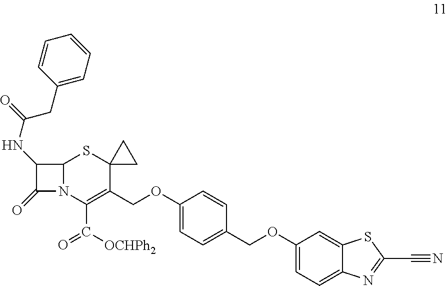

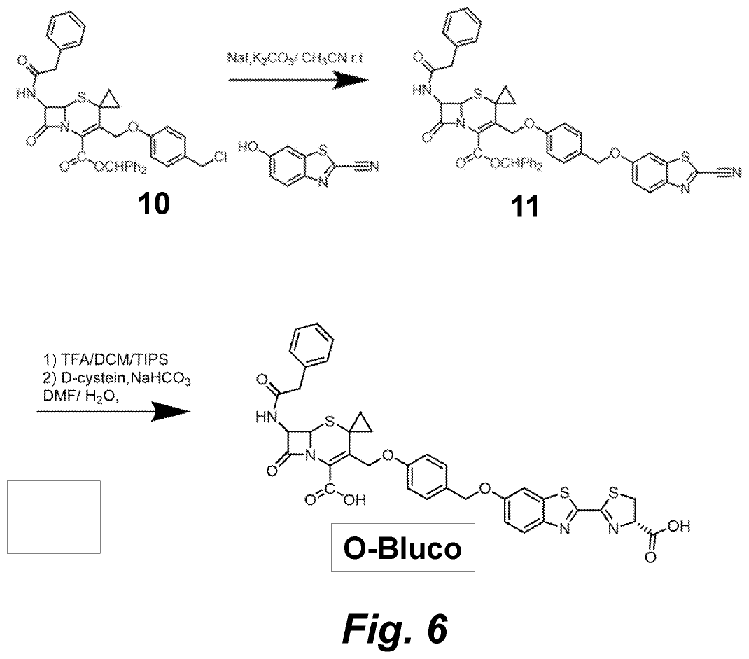

4-((4-(((2-cyanobenzo[d]thiazol-6-yl)oxy)methyl)phenoxy)methyl)-7-oxo-8-(2-phenylacetamido)-2-thia-6-azaspiro[bicyclo[4.2.0]octane-3,1′-cyclopropan]-4-ene-5-carboxylate (11). (Scheme 5, FIG. 6) A mixture of 6-hydroxybenzo[d]thiazole-2-carbonitrile (26.5 mg, 0.15 mmol), KCO₃ (20.7 mg, 0.15 mmol), NaI (11.2 mg, 0.075 mmol) in acetonitrile was stirred at room temperature for 10 min. Compound 1 (50 mg, 0.075 mmol) was then added to the above mixture and stirred at room temperature for 6 h. DCM was added and washed with water. Flash chromatography purification on silica gel column afforded (33.8 mg of compound 11 (45% yield). ¹H NMR (500 MHz, CDCl₃) δ 8.06 (d, J=9.0 Hz, 1H), 7.39-7.16 (m, 20H), 6.94 (s, 1H), 6.70 (d, J=8.4 Hz, 2H), 6.18 (d, J=8.7 Hz, 1H), 5.88 (dd, J=8.7, 4.8 Hz, 1H), 5.18 (d, J=4.8 Hz, 1H), 5.05 (s, 2H). 4.33 (d. J=1.8 Hz, 2H), 4.10 (d, J=7.1 Hz, 1H), 1.72 (s, 1H), 1.55-1.41 (m, 2H), 0.97-0.89 (m, 1H). ¹³C NMR (126 MHz, CDCl3) δ 164.92, 161.37, 159.50, 157.97, 146.99, 139.06, 138.78, 137.41, 133.60, 133.54, 129.50, 129.36, 129.27, 128.58, 128.49, 128.47, 128.42, 128.27, 128.00, 127.91, 127.82, 127.70, 126.98, 125.95, 119.06, 114.78, 113.25, 104.22, 79.54, 77.38, 77.12, 76.87, 70.47, 60.48, 59.85, 59.57, 43.28, 22.27, 21.29, 21.13, 13.62. LCMS: Found: 804.21

### Example 16

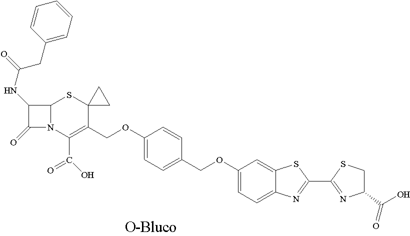 [SMILES: O=C(Cc1ccccc1)NC1C(=O)N2C(C(=O)O)=C(COc3ccc(COc4ccc5nc(C6=N[C@@H](C(=O)O)CS6)sc5c4)cc3)C3(CC3)SC12]

4-((4-(((2-((S)-4-carboxy-4,5-dihydrothiazol-2-yl)benzo[d]thiazol-6-yl)oxy)methyl)phenoxy)methyl)-7-oxo-8-(2-phenylacetamido)-2-thia-6-azaspiro[bicyclo[4.2.0]octane-3,1′-cyclopropan]-4-ene-5-carboxylic acid (O-Bluco): To a solution of 2 (10.3 mg, 0.016 mmol) in 1.5 mL of dry dichloromethane was added triisopropyl silane (TIPS, 20 μL) and trifluoroacetic acid (200 μL) at 0° C. The mixture was stirred for 1 h at the same temperature, then the solvent was evaporated under reduced pressure and the residue was washed with ether (1 mL×3) and the product was used for the next step without further purification. To a solution of crude product from previous step in 1.5 mL of DMF was added a solution of D-cysteine hydrochloride (7.4 mg, 0.047 mmol) and NaHCO₃ (6.6 mg, 0.078 mmol) in 0.5 mL of H₂O at room temperature. The reaction mixture was stirred for 20 min at room temperature. The product was purified with semi-prep HPLC (5.5 mg, 50% yield) as a white solid. HRMS: Calculated for C₃₆H₃₀N₄O₈S₃ ([M+H]⁺):743.12, Found: 743.1286.

¹H NMR (500 MHz, DMSO) δ 9.18 (d, J=8.2 Hz, 1H), 8.07 (d, J=9.0 Hz, 1H), 7.87 (d, J=2.5 Hz, 1H). 7.43 (d, J=8.5 Hz, 2H), 7.36-7.22 (m, 8H), 6.97 (d, J=8.6 Hz, 2H), 5.80 (dd, J=8.3, 4.8 Hz, 1H), 5.43 (dd, J=9.8, 8.3 Hz, 1H), 5.30 (d, J=4.8 Hz, 1H), 5.13 (s, 2H), 4.53 (d, J=11.0 Hz, 1H), 4.40 (d, J=11.3 Hz, 1H), 3.77 (d, J=10.4 Hz, 1H), 3.69 (dd, J=11.2, 8.3 Hz, 2H), 1.50 (s, 2H), 1.35 (d, J=9.5 Hz, 1H), 0.94 (s, 1H). ¹³C NMR (126 MHz, CDCl₃) δ 171.52, 165.21, 161.68, 159.84, 158.31, 147.35, 139.39, 139.10, 137.75, 133.90, 133.83, 129.87, 129.68, 128.91, 128.83, 128.81, 128.62, 128.34, 128.26, 128.24, 127.97, 127.33, 126.32, 119.39, 115.12, 113.57, 104.54, 79.88, 77.67, 77.41, 77.16, 70.82, 62.92, 60.17, 59.92, 43.70, 22.61, 21.62, 13.96.

We claim:

1. A caged bioluminescent probe comprising a luciferin moiety, an enzyme-cleavable moiety, and a caging moiety, wherein the moieties are conjugated to form the structure having the formula:
(Luciferin moiety)-(enzyme-cleavable moiety)-(caging moiety),
and wherein the enzyme-cleavable moiety is cleavable by a bacterial enzyme.

2. The caged bioluminescent probe of claim 1 further comprising a linker moiety between the luciferin moiety and the enzyme-cleavable moiety.

3. The caged bioluminescent probe of claim 1, wherein the enzyme-cleavable moiety is cleavable by a β-lactamase.

4. The caged bioluminescent probe of claim 1, wherein the caging moiety is a quencher.

5. The caged bioluminescent probe of claim 1, wherein the quencher is a diabcyl quencher.

6. The caged bioluminescent probe of claim 3, wherein the caged bioluminescent probe has the formula: 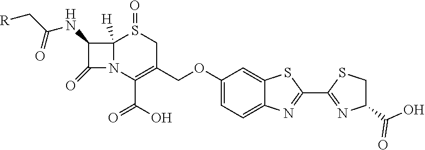
wherein R is

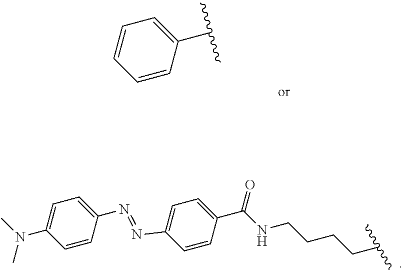 [SMILES: *c1ccccc1 ; *CCCCNC(=O)c1ccc(/N=N/c2ccc(N(C)C)cc2)cc1]

7. The caged bioluminescent probe of claim 1, wherein the enzyme-cleavable moiety is cleavable by a carbapenemase.

8. The caged bioluminescent probe of claim 7, wherein the caged bioluminescent probe has the formula: 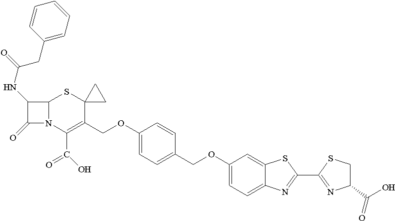 [SMILES: O=C(Cc1ccccc1)NC1C(=O)N2C(C(=O)O)=C(COc3ccc(COc4ccc5nc(C6=N[C@@H](C(=O)O)CS6)sc5c4)cc3)C3(CC3)SC12] 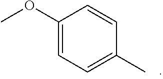
wherein the linker has the structure

9. The caged bioluminescent probe of claim 8, having the formula:

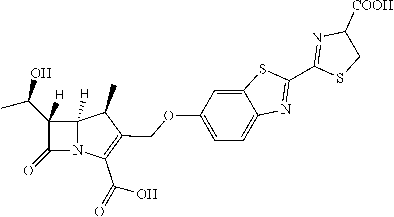 [SMILES: C[C@@H](O)[C@H]1C(=O)N2C(C(=O)O)=C(COc3ccc4nc(C5=NC(C(=O)O)CS5)sc4c3)[C@H](C)[C@H]12]

10. The caged bioluminescent probe of claim 9 further comprising a linker moiety between the luciferin moiety and the enzyme-cleavable moiety

11. The caged bioluminescent probe of claim 10, having the formula: 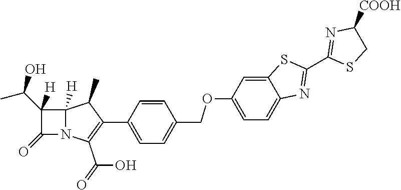 [SMILES: C[C@@H](O)[C@H]1C(=O)N2C(C(=O)O)=C(c3ccc(COc4ccc5nc(C6=N[C@@H](C(=O)O)CS6)sc5c4)cc3)[C@H](C)[C@H]12] 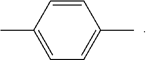
wherein the linker has the structure

12. A method of identifying a bacterial strain resistant to a β-lactam antibiotic or a carbapenem antibiotic, wherein said method comprises contacting a population of bacteria with a caged bioluminescent probe cleavable by either a β-lactamase or a carbapenemase. adding luciferinase; and measuring an emitted bioluminescent signal, wherein a detected emitted bioluminescent signal indicates that the bacterial strain has a β-lactamase or a carbapenemase activity.

13. The method of claim 12, wherein the caged bioluminescent probe comprises a luciferin moiety, an enzyme-cleavable moiety, and a caging moiety, wherein the moieties are conjugated to form the structure having the formula:
(luciferin moiety)-(enzyme-cleavable moiety)-(caging moiety),
and wherein the enzyme-cleavable moiety is cleavable by a bacterial enzyme.

14. The method of claim 12, wherein the caged bioluminescent probe comprises a linker moiety between the luciferin moiety and the enzyme-cleavable moiety.

15. The method of claim 12, wherein the enzyme-cleavable moiety is cleavable by a β-lactamase or by a carbapenemase.

16. The method of claim 12, wherein the caging moiety is a quencher.

17. The method of claim 12, wherein the caged bioluminescent probe has the formula: 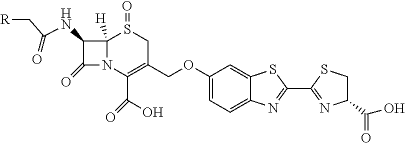 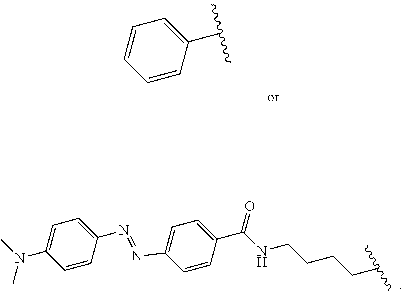 [SMILES: *c1ccccc1 ; *CCCCNC(=O)c1ccc(/N=N/c2ccc(N(C)C)cc2)cc1]
wherein R is

18. The method of claim 15, wherein the caged bioluminescent probe has the formula: 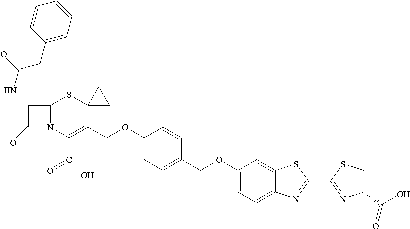 [SMILES: O=C(Cc1ccccc1)NC1C(=O)N2C(C(=O)O)=C(COc3ccc(COc4ccc5nc(C6=N[C@@H](C(=O)O)CS6)sc5c4)cc3)C3(CC3)SC12] 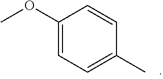
wherein the linker has the structure and wherein the enzyme-cleavable moiety is cleavable by a carbapenemase.

19. The method of claim 12, wherein the caged bioluminescent probe has the formula:

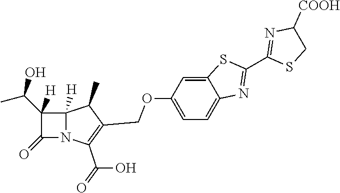 [SMILES: C[C@@H](O)[C@H]1C(=O)N2C(C(=O)O)=C(COc3ccc4nc(C5=NC(C(=O)O)CS5)sc4c3)[C@H](C)[C@H]12]

20. The method of claim 19, wherein the caged bioluminescent probe further comprising a linker moiety between the luciferin moiety and the enzyme-cleavable moiety.

21. The method of claim 20, wherein the caged bioluminescent probe has the formula: 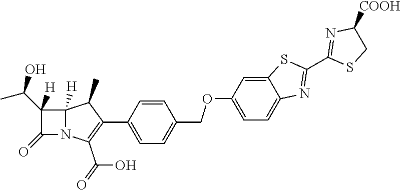 [SMILES: C[C@@H](O)[C@H]1C(=O)N2C(C(=O)O)=C(c3ccc(COc4ccc5nc(C6=N[C@@H](C(=O)O)CS6)sc5c4)cc3)[C@H](C)[C@H]12] 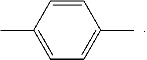
wherein the linker has the structure
